(19) United States
(12) Patent Application Publication
(10) Pub. No.: US 2004/0229908 A1
(43) Pub. Date: Nov. 18, 2004

(54) Compositions and methods for the treatment of Parkinson's disease and tardive dyskinesias

(76) Inventor: Jodi Nelson, Denver, CO (US)

Correspondence Address: GREENLEE WINNER AND SULLIVAN P C, 5370 MANHATTAN CIRCLE, SUITE 201, BOULDER, CO, 80303, US

(21) Appl. No.: 10/616,692

(22) Filed: Jul. 9, 2003

Related U.S. Application Data
(63) Continuation-in-part of application No. 10/192,414, filed on Jul. 9, 2002.
Application No. 10/192,414 is a continuation-in-part of application No. 09/615,639, filed on Jul. 13, 2000, now Pat. No. 6,417,177.
(60) Provisional application No. 60/143,767, filed on Jul. 13, 1999.
(60) Provisional application No. 60/175,051, filed on Jan. 7, 2000.
(60) Provisional application No. 60/202,140, filed on May 5, 2000.
(60) Provisional application No. 60/479,748, filed on Jun. 19, 2003.

(51) Int. Cl. A61K 31/47

(52) U.S. Cl. 514/313

(57) ABSTRACT

This invention provides compositions and methods for increasing cellular respiration of melanized catecholamine neurons, and methods for alleviating symptoms or stopping appearance and/or progression of symptoms of Parkinson's disease and related conditions, characterized by nigrostriatal degeneration, as well as drug-induced dyskinesias, tardive dyskinesia, Neuroleptic Malignant Syndrome, and negative symptoms of schizophrenia. An effective amount of a neuromelanin-binding composition having a quinoline ring in a suitable pharmaceutical carrier is administered to patient in need of such treatment. Preferably the composition comprises (−)-chloroquine diphosphate. Selected adjuvants are also provided as part of the compositions of this invention.

This application is a continuation-in-part of U.S. patent application Ser. No. 10/192,414 filed Jul. 9, 2002, which is a continuation-in part of U.S. patent application Ser. No. 09/615,639 filed Jul. 13, 2000, now U.S. Pat. No. 6,417,177 issued Jul. 9, 2002, which takes priority from U.S. Patent Application No. 60/143,767 filed Jul. 13, 1999, U.S. Patent Application No. 60/175,051 filed Jan. 7, 2000, and U.S. Patent Application No. 60/202,140 filed May 5, 2000. This application also takes priority from U.S. Provisional Patent Application Ser. No. 60/479,748 filed Jun. 19, 2003. All of the foregoing applications are incorporated herein by reference to the extent not inconsistent herewith.

### BACKGROUND

Idiopathic Parkinson's Disease (IPD) is a progressive neurodegenerative disorder. The onset of IPD symptoms begin to manifest when a threshold reduction of 60%-70% nigral neurons accompanied by an 80%-90% attenuation in striatal dopamine efflux, has been reached (Koller, W. C., “When does Parkinson's disease begin?” (1992) Neurology 42(S4):27-31). Symptoms include tremor, postural imbalance, rigidity, bradykinesia and akinesia (Diagnostic Clinical Neuropsychology, Bigler, E. and Clement, P., Eds., 3^{rd} Ed. 1997). These symptoms intensify as the disease progresses. In severe stages of IPD, following the onset of akinesia, even the simplest movements require a monumental degree of concentration and mental effort, often to the point of anguish (Textbook of Medical Physiology, Guyton, A. C. and Hall, J. E., Eds., 9^{th} Ed., W. B. Saunders Company, Philadelphia, Pa., 1996). IPD is also characterized by a number of autonomic (Vainshtok, A. B., “Treatment of Parkinsonism with delagil,” (1972) Klin. Med (Mosk) 50(9):51-56) and non-motor symptoms including depression (Cummings, J. L., “Depression and Parkinson's Disease: A Review,” (1992) Am. J Psychiatry 149(4):443-454) and frontal lobe dysfunction (Gotham, A. M. et al., “Levodopa treatment may benefit or impair ‘frontal’ function in Parkinson's disease,” (1986) Lancet 25;2(8513):970-971).

In the United States, it is estimated that 5-24 in every 100,000 people suffer from IPD, with the majority of low-income cases going undiagnosed (Chrischilles, E. A. et al., “The health burdens of Parkinson's disease,” (1998) Movement Disorders 13(3):406-413). In 1995, the World Health Organization (WHO) conducted a global epidemiological evaluation of the incidence of IPD, showing a worldwide incidence of 5.32 per 100,000 people with an astounding incidence rate of 49.33 per 100,000 people over the age of 65 (M. Privett, WHO). Although more recent epidemiological figures are unavailable, in 1996 with the world population being approximately 5.7 billion, an estimated 2.8 million people had a confirmed diagnosis of IPD.

Current pharmacological treatments for IPD and other Parkinsonian-like motor disorders include anticholinergic agents, catechol-o-methyltransferase inhibitors and dopaminergic agents (Physicians' Desk Reference, 2000, 54^{th} Ed., Medical Economics Company, Inc., Montvale, N.J.). Since the late sixties, dopamine precursor L-DOPA has been employed for the symptomatic relief of IPD motor dysfunction (Mena, M. A. et al., “Pharmacokinetics of L-DOPA in patients with Parkinson's disease,” (1986) Advances in Neurology 45:481-486). However, following long-term use of L-DOPA (generally 5-8 years), diminished therapeutic efficacy is observed in approximately 50% of IPD patients (Roos, R. A. et al., “Response fluctuations in Parkinson's disease,” (1990) Neurology 40(9):1344-1346). A wearing off of L-DOPA efficacy precedes the development of serious motor side effects such as on/off motor oscillations and dyskinesias (Carlsson, Arvid, “Development of new pharmacological approaches in Parkinson's disease,” (1986) Advances in Neurology 45:513-518). Further, when medications are increased to compensate for the development of these new motor dysfunctions, more serious side effects are generally observed, including psychiatric complications, while producing only minimal therapeutic benefit (Stoof, J. C. et al., “Leads for the development of neuroprotective treatment in Parkinson's disease and imaging methods for estimating treatment efficacy,” (1999) Eur. J Pharmacol. 375(1-3):75-86).

Up to 20% of the people initially diagnosed with IPD actually suffer from atypical IPD (APD), striatonigral degeneration (SND), or multiple symptom atrophy (MSA) (Antonini, A. et al., “Differential diagnosis of Parkinsonism with [¹⁸F]Fluorodeoxyglucose and PET,” (1998) Movement Disorders 13(2):268-274). Little or no response to conventional Parkinson's disease drug therapy is usually the differentiating factor between a diagnosis of APD, SND and MSA as opposed to IPD (Dethy, S. et al., “Asymmetry of basal ganglia glucose metabolism and L-dopa responsiveness in Parkinsonism,” (1998) Movement Disorders 13(2):275-280). Often, little can be done for people suffering these atypical afflictions. Therefore, it would be of great benefit if a pharmacological means were identified that could alleviate symptoms of atypical Parkinson's disease, as well as IPD.

Schizophrenia, affecting approximately 1% of persons over the age of eighteen, is by far the most costly and debilitating mental illnesses within and in many countries outside of the United States (Rupp, A. and Keith, S. J. (1993), “The cost of schizophrenia: assessing the burden,” Psych Clin N Am, 16:413-423). Direct treatment expenditures exceeding 30 billion were reported in 2000. The majority of the costs for treating this disorder are paid predominately by governmental sources, including Medicaid and Medicare (Martin, B. C. et al. (2001), “Antipsychotic prescription use and costs for persons with schizophrenia in the 1990s: current trends and five year time series forecasts,” Schizo Res, 47(2-3):281-292). With current budget cuts/reductions being implemented to these programs, healthcare provides are consigned to administer their patients diagnosed with schizophrenia and other affective disorders the less costly “traditional” antipsychotic medications including phenothiazines and decanoates, such as haloperidol and fluphenazine (Laurie Lucero, LCSW, CACIII, personal communication) which are dopamine antagonists and are just as, if not more, effective in alleviating psychosis than the newer atypical medications, such as Clozapine, Risperidone and Olanzapine (Seeman, P. and Kaput, S. (1997), “Clozapine occupies high levels of dopamine d2 receptors,” Life Sci, 60(12):207-216). However, these older medications often produce more side effects and have a higher dyskinetic profile than the newer, more expensive atypical neuroleptics (Martin, B. C. et al. (2001), “Antipsychotic prescription use and costs for persons with schizophrenia in the 1990s: current trends and five year time series forecasts,” Schizo Res, 47(2-3):281-292).

Drug-induced movement disorders (DIDs) pose a serious problem to physicians, complicating the successful effective pharnacotherapeutic treatment of patients diagnosed with schizophrenia, Parkinson's Disease and other dopamine-associated disorders (Rodnitzky, R. L. (2002), “Drug-induced movement disorders,” Clin Neuropharmacol, 25(3):142-152). Similar to what is observed in Parkinson's disease during dopamine agonist treatment, following approximately five years of dopamine antagonist therapy, 20-25% of patients with schizophrenia begin to manifest tardive dyskinesia (Morgenstern, H. and Glazer, W. M. (1993), “Identifying risk-factors for tardive dyskinesia among long-term outpatients maintained with neuroleptic medications,” Arch Gen Psych, 50:723-733); Kane, J. M. (1995), “Tardive dyskinesias: epidemiological and clinical presentation,” In: Psychopharmacology: The Fourth Generation of Progress, Bloom, R. E. and Kupfer, D. J., Eds., New York, N.Y., Raven Press, pp. 1485-1496). The incidence of developing tardive dyskinesias during neuroleptic treatment increases with use. A reported 49% prevalence rate is seen in persons who have taken neuroleptics for ten years, which increases to 68% if a person has been taking them for 25 years (Glazer, W. M. et al. (1993), “Predicting the long-term risk of tardive dyskinesia in out-patients maintained on neuroleptic medications,” J Clin Psych, 54(4):133-139).

Tardive dyskinesias are characterized by similar dyskinetic states (e.g., chorea, dystonia, etc) as are seen in Parkinson's patients experiencing levodopa-induced dyskinesias (LIDs) (Nutt, J. G. (2000), “Clinical pharmacology of levodopa-induced dyskinesia,” Ann. Neurol., 47(suppl 1):S160-S166). Neuroleptic Malignant Syndrome (NMS), which is often mistakenly associated with schizophrenia and neuroleptic medications, also affects Parkinson's patients, manifesting most frequently following dopaminergic dose reductions, drug withdrawal, and in sensitive patients during dopaminergic “wearing-off” periods between dosages. Motor symptoms of NMS include axial rigidity, dystonia, chorea, Parkinsonisms and oral-bucco-facial dyskinesias, which appear to be predominately mediated via the dopamine system (Hansen, T. E. et al., (1997), “Neuroleptic intolerance,” Schizo. Bull. 23(4):567-582). However, altered consciousness, hypothermia, autonomic nervous system instability and respiratory disturbance pose a greater threat to the morbidity of an individual, and thus are considered more important than motor symptoms of NMS (Rodnitzky, R. L. (2002), “Drug-induced movement disorders,” Clin Neuropharmacol, 25(3):142-152).

It has long been recognized that during the course of levodopa (L-dopa) therapy that a predominant number of patients develop motor fluctuations (MFs) (Chase, T. N. et al. (1986), “Fluctuation in response to chronic levodopa therapy pathogenetic and therapeutic considerations,” Adv Neurol, 45:477-480) and/or levodopa-induced dyskinesias (Klawans, H. and Shenker, D. (1970), “Theoretical implications of the use of L-dopa in Parkinsonism,” Acta Neurol Scand, 46:409-441). Bergmann and colleagues (Bergmann, K. J., et al. (1986), “Parkinson's disease and long term levodopa therapy,” Adv Neurol, 45:463-467) longitudinally evaluated a group of 295 Parkinson's patients who had been initiated on L-dopa therapy. Of the 295 patients, 263 (93%) developed dyskinesias and 163 (58%) of these patients began to experience daily motor fluctuations within several years of L-dopa introduction. Motor fluctuations frequently occur in persons having early onset Parkinson's Disease (PD); whereas, levodopa-induced dyskinesias commonly manifest early in cases of severe Parkinson's disease. It has recently been established that 15%-30% of patients undergoing embryonic putamen cell transplants develop “run away” dyskinesias even in the absence of L-dopa or dopaminergic drug therapy (Michael J. Fox Foundation, 2003). The mechanisms underlying these disorders have been thought to be unrelated (Textbook of Medical Physiology, (1996), 9^{th} Guyton, E. and Hall, A.; John E. W.B. Saunders Company Philadelphia, Pa.; Diagnostic Clinical Neuropsychology, (1997), 3^{rd} Ed. Bigler, Erin Clement, Pamelia, University of Texas Press Austin, Tex.).

Methods for treating these dyskinesias are needed.

Chloroquine Compounds

Chloroquine [7-chloro-4-(4-diethylamino-1-methylbutylamino)quinoline] (The Merck Index, p. 2220, 1996) is a synthetically manufactured anti-malarial containing the quinoline nucleus. Chloroquine was developed over fifty years ago. It continues to be the most widely employed drug for the treatment of the asexual erythrocytic form of P. falciparum (Deepalakshmi, P. D. et al., “Effect of chloroquine on rat liver mitochondria,” (1994) Indian J Exp. Biology 32(11):797-799). A number of chloroquine derivatives have been identified for antimalarial and other uses. See U.S. Pat. Nos. 5,948,791, 5,834,505, 5,736,557, 5,639,737, 5,624,938, 5,596,002 and 4,421,920.

Enantiomers of Chloroquine

Chloroquine and hydroxychloroquine are racemic mixtures of (−)- and (+)-enantiomers. The (−)-enantiomers are also known as (R)-enantiomers (physical rotation) and 1-enantiomers (optical rotation). The (+)-enantiomers are also known as (S)-enantiomers (physical rotation) and d-enantiomers (optical rotation). The (+)-enantiomer metabolizes peripherally about eight times more rapidly than the (−)-enantiomer, producing toxic metabolites including de-ethyl chloroquine (Augustijins, P. and Verbeke, N. [1993] “Stereoselective pharmacokinetic properties of chloroquine and de-ethyl chloroquine in humans,” Clinical Pharmacokinetics 24(3):259-69; Augustijins, P. et al. [1999], “Stereoselective de-ethylation of chloroquine in rat liver microsomes,” Eur. J Drug Metabolism & Pharmacokinetics 24(1):105-8; DuCharme, J. and Farinotti R. [1996], “Clinical pharmacokinetics and metabolism of chloroquine,” Clinical Pharmacokinetics 31(4):257-74). Administering (+)-chloroquine may cause cardiac side effects due to toxic metabolite formation. The (−)-enantiomer has a longer half-life and lower clearance than the (+)-enantiomer (Ducharme, J. et al. [1995), “Enantio-selective disposition of hydroxychloroquine after a single oral dose of the racemate to healthy subjects,” British J Clinical Pharmacology 40(2): 127-33). The enantiomers of chloroquine and hydroxychloroquine may be prepared by procedures known to the art.

All publications referred to herein are incorporated by reference to the extent not inconsistent herewith.

### SUMMARY OF THE INVENTION

This invention provides compositions and methods for increasing cellular respiration of melanized catecholamine neurons such as dopamine neurons in the substantia nigra and basal ganglion, epinephrine and norepinephrine neurons, of protecting such neurons against oxidative stress, excitotoxicity, and apoptosis.

The compositions of this invention are useful for treatment of Parkinson's Disease and related conditions, including cognitive symptoms of Parkinson's disease, Drug-Induced Dyskinesias, Tardive Dyskinesia, as well as Negative Symptoms of Schizophrenia and related conditions, including both alleviation of symptoms and preventing onset or progression of symptoms of these conditions. The compositions of this invention are also useful for treating Neuroleptic Malignant Syndrome (NMS), which afflicts persons taking dopamine precursors, dopamine agonists and/or neuroleptic medications, and for treating motor fluctuations associated with use of dopamine precursors or agonists to treat movement disorders. The compositions of this invention may be administered long-term.

The compositions of this invention are also useful for prolonging the utility and efficacy of L-Dopa, and dopamine agonists which temporarily or permanently lose their ability to ameliorate the symptoms of Parkinson's disease after an initial period of effectiveness. The compositions of this invention are also useful for improving the safety and tolerability profile of “typical” neuroleptic medications, such as phenothiazines and decanoates, which promote tardive dyskinesia and contribute to the development of negative symptoms of schizophrenia.

The term “Parkinson's Disease and related conditions” as used herein includes idiopathic Parkinson's Disease (IPD), atypical Parkinson's Disease (APD), non-L-dopa-responsive atypical Parkinson's Disease, Parkinson's Plus syndromes (which include supranuclear palsy and other non L-dopa responsive Parkinson's-type diseases), striatonigral degeneration (SND), multiple symptom atrophy (MSA), and vascular Parkinson's Disease and dystonia.

The term “Drug induced dyskinesias” as used herein includes hypokinetic conditions and disorders, such as Parkinson's Disease and related conditions, Drug-Induced Parkinsonism (DID), Extra Pyramidal Disorders (EPS) and akathisia. The term “Drug induced dyskinesias” as used herein also includes hyperkinetic conditions and disorders, such as Levodopa-Induced Dyskinesia (LID), Tardive Dyskinesia (TD), chorea and ballisms.

The term “treating” with respect to a condition described herein means alleviating symptoms or stopping appearance and/or progression of symptoms.

Tardive dyskinesias from long-term use of therapeutic drugs are movement disorders that develop after a number of years, e.g. five to eight years, of taking “typical” neuroleptic therapeutic drugs.

For example, neuroleptic-induced tardive dyskinesias, a hyperkinetic condition due to neural degeneration, often develop after a period of several years in patients taking “typical” neuroleptic drugs such as chlorpromazine, fluphenazine and haloperidol. In addition, Levodopa-induced dyskinesia (LID) is a hyperkinetic disorder that typically develops in Parkinson's patients after several years of taking Levodopa for Parkinson's or other movement disorders such as Wilson's disease. Clinical studies reported herein have shown good improvement in hyperkinetic dyskinesias in Parkinson's patients after taking chloroquine diphosphate.

Clinical studies reported herein using the chloroquine diphosphate study drug, in addition to demonstrating reduction of Parkinson's movement, also demonstrate reduction of cognitive symptoms of Parkinson's disease such as memory loss, difficulty in word finding and loss of ability to multitask.

Motor fluctuations associated with the use of dopamine or dopamine agonists for the treatment of various movement disorders occur because such drugs are usually administered in periodic doses. Typically, the patient experiences an “ON” state shortly after administration of the drug, and then when the drug begins to leave the system the patient turns “OFF”, wherein movement again becomes difficult again. Due in part to the long half-life of the active ingredients of this invention, “ON/OFF” motor functions seen in patients taking dopamine precursors and/or dopamine agonists are stabilized.

Negative symptoms of schizophrenia include apathy, loss of verbal fluency, affective flattening, lack of motivation, and depression. These symptoms are the result of neurodegenerative effects and can be ameliorated by dopaminergic agents. Therefore, the compositions of this invention are useful for treating the negative symptoms of schizophrenia because of their dopaminergic effects. Additionally, the compositions of this invention are useful for preventing the manifestation and/or progression of the negative symptoms of schizophrenia because of their neural-protective effects. For this purpose an appropriate dosage will be that used for treatment of Parkinson's disorders. The compositions of this invention reduce oxidative stress on neurons, intercalate and stabilize DNA, and promote and/or maintain the expression of brain-derived neurotrophic factors that protect neurons against apoptosis, thereby preventing or reducing degeneration of these neurons.

The compositions of this invention are also useful for selectively inducing increased amounts of glial-derived neurotrophic factor (GDNF) in areas of the brain where its presence has a beneficial effect on movement disorders such as Parkinson's disease. It has been found that although systemic administration of GDNF to Parkinson's patients does not have a beneficial effect, injection of GDNF directly into the putamen did have a beneficial effect. This beneficial effect can be achieved without the necessity for such injection by preventing the activation of inhibitory factor kappa B-α (IFkappaB-α), the molecule responsible for deactivating nuclear factor kappa B (NFkappaB) that promotes the synthesis of GDNF in areas of the brain affecting Parkinson's. These areas of the brain include, but are not limited to the substantia nigra, striatum (putamen, caudate and areas of the nucleus accumbens), and the globus pallidus (internal and external segments). The term “selectively” is used in this context to indicate that comparatively little of the GDNF (less than about one-tenth as much) is induced in the cerebral cortex where the compositions of this invention do not accumulate, “target” or exert a therapeutic effect.

The compositions of this invention are also useful for reducing apoptosis in melanized catecholamine and other neurons contained in the striatum, basal ganglia, mesencephalon, brain stem and cerebellum by mechanisms elucidated hereinafter, thereby providing neural protective effects. “Reducing apoptosis” means measurably changing a parameter in a way that is indicative of reduced apoptosis, such as reduced levels of free radicals and pro-apoptotic cytokines, increased production of anti-apoptotic molecules (via inhibition of NFkappaB inactivation by IFkappaBcα) and reductions in cell shrinking and DNA fragmentation.

The compositions of this invention are also useful for reducing thalamic hyperactivity in patients experiencing hyperkinetic states. These compositions modulate the thalamic motor relays in the ventral anterior, ventral lateral and reticularis nucleus portions of the thalamus. Modulation rather than complete inhibition of activity of these motor relays leads to improved motor function. “Modulation of thalamic activity” and “reduction of thalamic hyperactivity” are measured by means known to the art, preferably by showing measurably improvement in motor function.

Compositions of this invention include the active ingredients described herein combined with dopamine precursors and dopamine agonists. It has been found in clinical studies that the dosage of dopamine and dopamine agonists used to treat movement disorders may be reduced by about one-half when combined with the active ingredients hereof. Typically this means that the dopamine or dopamine agonists do not need to be administered as many times during the day, although the amount of dopamine or dopamine agonist administered each time may also be reduced. The ratio of active ingredient hereof to dopamine or dopamine agonist in these compositions should be about 5:95 to about 25:95. In later-stage patients who require higher dosages of dopamine or dopamine agonists, the ratio of active ingredient to dopamine or dopamine agonist should be lower than in earlier-stage patients.

For patients in whom dopaminergic effects do not need to be avoided, such as patients with damaged dopamine neurons, appropriate dosage levels of chloroquine and related compounds are as set forth herein for treatment of Parkinson's disease. For patients in whom dopaminergic effects are contraindicated, dosages should be lower, as may be readily determined by those skilled in the art without undue experimentation.

Compositions of this invention include the active ingredients described herein combined with traditional phenothiazine and/or decanoate neuroleptics, to improve the safety and tolerability of these agents while preventing the permanent neurological damage resulting from long term use of these drugs, which leads to the formation of tardive dyskinesia and negative symptoms of schizophrenia. Traditional phenothiazine neuroleptics include Chlorpromazine, Chlorprothixene, Fluphenazine, Haloperidol, Loxapine, Mesoridazine, Molindone, Perphenazine, Pimozide, Prochlorperazine, Promazine, Thioridazine, Thiothixene, and Trifluoperazine. Decanoate neuroleptics include Fluphenazine decanoate and Haloperidol decanoate. The amount of choroquine or related compound should be less than that which exerts a dopaminergic effect, preferably the amount is between about 25 and 100 mg per day.

This invention provides compositions useful for the foregoing purposes comprising an active ingredient as described below, racemic mixtures, and enantiomers thereof, preferably covalently linked, mixed, or complexed with an adjuvant, and acceptable pharmaceutical salts thereof, and mixtures of the foregoing. The active ingredient and adjuvant (if used) should be present in amounts effective to provide a function selected from the following: increase cellular respiration of melanized catecholamine neurons, exert a dopaminergic effect, inhibit the production of pro-inflammatory cytokines and interleukins, intercalate DNA, antagonize acetylcholine receptors in the substantia nigra, striatum and nucleus of the thalamus, inactivate NMDA receptor subunits NRA2A and NRA2B, and promote the synthesis if GDNF.

The compositions of this invention comprise an active ingredient as described below, racemic mixtures, and enantiomers thereof, covalently linked, mixed, or complexed with an adjuvant, acceptable pharmaceutical salts thereof, and mixtures of the foregoing, said active ingredient and adjuvant being present in amounts effective to increase melanized catecholamine neurons.

The active ingredient is preferably chloroquine or a related compound (referred to herein as “CQ.” The term “CQ” includes chloroquine (7-chloro-4-(4-diethylamino-1-methylbutylamino)quinoline), chloroquine phosphate (7-chloro-4-(4-diethylamino-1-methylbutylamino) quinoline phosphate, and hydroxychloroquine (7-chloro-4-(4-diethylamino-1-methylbutylamino) quinoline), racemic mixtures, enantiomers, suitable pharmaceutical salts thereof, and mixtures thereof. Similarly, the terms (−)-chloroquine and (+)-chloroquine include (−)- and (+)-chloroquine phosphate and (−)- and (+)-hydroxychloroquine respectively.

The active ingredient may also be selected from compounds as above wherein hydrogen or fluorine is substituted for the chlorine atom on the molecule, e.g., 7-fluoro-4-(4-diethylamino-1-methylbutylamino)quinoline, 4-(4-diethylamino-1-methylbutylamino)quinoline, 7-fluoro-4-(4-diethylamino-1-methylbutylamino) quinoline phosphate, 4-(4-diethylamino-1-methylbutylamino) quinoline phosphate 7-fluoro-4-(4-diethylamino-1-methylbutylamino quinoline, and 4-(4-diethylamino-1-methylbutylamino quinoline, racemic mixtures, enantiomers, suitable pharmaceutical salts thereof, and mixtures thereof. Similarly, the terms (−)-enantiomer and (+)-enantiomer include (−)- and (+)-enantiomer phosphate and (−)- and (+)-hydroxy analogs of the foregoing respectively.

Compositions useful for increasing cellular respiration of melanized catecholamine neurons, and/or alleviating, preventing or halting progress of Parkinson's symptoms also may comprise, as active ingredients, neuromelanin-binding chloroquine and fluorine analogs and derivatives containing a quinoline nucleus, preferably selected from the group consisting of:

7-chloro-4-(4-diethylamino-1-methylbutylamino)quinoline (chloroquine);

7-fluoro-4-(4-diethylamino-1-methylbutylamino)quinoline;

4-(4-diethylamino-1-methylbutylamino)quinoline;

7-hydroxy-4-(4-diethylamino-1-methylbutylamino)quinoline;

chloroquine phosphate;

7-chloro-4-(4-diethylamino-1-butylamino)quinoline (desmethylchloroquine);

7-fluoro-4-(4-diethylamino-1-butylamino)quinoline);

4-(4-diethylamino-1-butylamino)quinoline;

7-hydroxy-4-(4-diethylamino-1-butylamino)quinoline;

7-chloro-4-(1-carboxy-4-diethylamino-1-butylamino)quinoline;

7-fluoro-4-(1-carboxy-4-diethylamino-1-butylamino)quinoline;

4-(1-carboxy-4-diethylamino-1-butylamino)quinoline;

7-hydroxy-4-(1-carboxy-4-diethylamino-1-butylamino)quinoline;

7-chloro-4-(1-carboxy-4-diethylamino-1-methylbutylamino)quinoline;

7-fluoro-4-(1-carboxy-4-diethylamino-1-methylbutylamino)quinoline;

4-(1-carboxy-4-diethylamino-1-methylbutylamino)quinoline;

7-hydroxy-4-(1-carboxy-4-diethylamino-1-methylbutylamino)quinoline;

7-chloro-4-(4-ethyl-(2-hydroxyethyl)-amino-1-methylbutylamino)quinoline (hydroxychloroquine);

7-fluoro-4-(4-ethyl-(2-hydroxyethyl)-amino-1-methylbutylamino)quinoline;

4-(4-ethyl-(2-hydroxyethyl)-amino-1-methylbutylamino)quinoline

7-hydroxy-4-(4-ethyl-(2-hydroxyethyl)-amino-1-methylbutylamino)quinoline;

hydroxychloroquine phosphate;

7-chloro-4-(4-ethyl-(2-hydroxyethyl)-amino-1-butylamino)quinoline (desmethylhydroxychloroquine);

7-fluoro-4-(4-ethyl-(2-hydroxyethyl)-amino-1-butylamino)quinoline;

4-(4-ethyl-(2-hydroxyethyl)-amino-1-butylamino)quinoline;

7-hydroxy-4-(4-ethyl-(2-hydroxyethyl)-amino-1-butylamino)quinoline;

7-chloro-4-(1-carboxy-4-ethyl-(2-hydroxyethyl)-amino-1-butylamino)quinoline;

7-fluoro-4-(1-carboxy-4-ethyl-(2-hydroxyethyl)-amino-1-butylamino)quinoline;

4-(1-carboxy-4-ethyl-(2-hydroxyethyl)-amino-1-butylamino)quinoline;

7-hydroxy-4-(1-carboxy-4-ethyl-(2-hydroxyethyl)-amino-1-butylamino)quinoline;

7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline;

4-(1-carboxy-4-ethyl-(2-hydroxyethyl)-amino-1-methylbutylamino)quinoline;

7-hydroxy-4-(1-carboxy-4-ethyl-(2-hydroxyethyl)-amino-1-methylbutylamino)quinoline;

8-[(4-aminopentyl)amino]-6-methoxydihydrochloride quinoline;

1-acetyl-1,2,3,4-tetrahydroquinoline; 8-[4-aminopentyl)amino]-6-methoxyquinoline dihydrochloride;

1-butyryl-1,2,3,4-tetrahydroquinoline; 7-chloro-2-(o-chlorostyryl)-4-[4-diethylamino-1-methylbutyl]aminoquiinoline phosphate;

3-chloro-4-(4-hydroxy-α,α′-bis(2-methyl-1-pyrrolidinyl)-2,5-xylidinoquinoline, 4-[(4-diethylamino)-1-methylbutyl)amino]-6-methoxyquinoline;

3-fluoro-4-(4-hydroxy-α,α′-bis(2-methyl-1-pyrrolidinyl)-2,5-xylidinoquinoline, 4-[(4-diethylamino)-1-methylbutyl)amino]-6-methoxyquinoline;

4-(4-hydroxy-α,α′-bis(2-methyl-1-pyrrolidinyl)-2,5-xylidinoquinoline, 4-[(4-diethylamino)-1-methylbutyl)amino]-6-methoxyquinoline;

3,4-dihydro-1-(2H)-quinolinecarboxyaldehyde;

1,1′-pentamethylenediquinoleinium diiodide; and 8-quinolinol sulfate, racemic mixtures, and enantiomers thereof, and suitable pharmaceutical salts thereof including phosphate salts of all the foregoing compounds, said compounds covalently linked or complexed or mixed with adjuvants and mixtures thereof, as well as suitable pharmaceutical salts thereof. Chloroquine and hydroxychloroquine are preferred; (−)-enantiomers thereof are more preferred, and said compounds covalently linked or complexed or mixed with adjuvants are most preferred. Neuromelanin-binding compounds such as chlorpromazine and other antipsychotics, which bind to dopamine receptors, are not included within the scope of PD-effective neuromelanin-binding compounds of this invention. Any chloroquine analog or derivative known to the art and capable of binding neuromelanin may be useful in the methods of this invention.

The neuromelanin-binding compound may be selected from the group consisting of compounds capable of crossing the blood-brain barrier in effective amounts. Such compounds include those which are more lipophilic, are capable of changing to effective chirality after crossing the blood-brain barrier, have side chain substituents which enhance compound transport via blood-brain barrier transporter mechanisms, or are complexed or covalently linked with antibodies or other targeting moieties, or administered in combination with other compounds facilitating their crossing the blood-brain barrier, as known to the art. The (−)-enantiomer of chloroquine (referred to herein as the active enantiomer) is preferred.

The (−) enantiomers of chloroquine and related compounds intercalate with DNA of neural cells and protect the guanines which are otherwise subject to free radical attack leading to neural degeneration.

In a preferred embodiment, the compositions of this invention which are useful for increasing cellular respiration of melanized catecholamine neurons comprise an effective amount of a composition comprising (−)-CQ or (−)-CQ mixed, complexed, or covalently linked with an adjuvant; an amount of (+)-CQ less than that of said (−)-CQ or (−)-CQ mixed, complexed, or covalently linked with a targeting agent; and a suitable pharmaceutical carrier. When CQ enantiomers are administered separately, there is significantly less CQ accumulation in the eyes, and thus less CQ-associated retinal degeneration.

Such compositions containing (−)-chloroquine may include anywhere from no (+)-CQ to about 49% (+)-CQ. An amount of (+)-CQ sufficient to bind to enzymes causing peripheral breakdown of CQ is preferred, leaving more of the (−)-CQ to cross the blood brain barrier where its therapeutic effect takes place. Preferably the compositions comprise between about 10% and about 20% (+)-CQ.

Adjuvants herein are preferably selected from the group consisting of peripheral membrane protective agents, such as retinal protective agents, peripheral metabolism inhibitors which inhibit peripheral metabolism of the active ingredient, enhancing agents such as histamine H₁ receptor antagonists, neural protective compounds other than the active ingredients as defined herein, dopamine and dopamine agonists, free radical deactivators, and antioxidants.

A targeting agent is a substance that when complexed with the active ingredient helps carry it across the blood brain barrier. Preferred targeting agents are lipophilic moieties known to the art which are attached to the active molecule at a position which does not interfere with the ability of the quinoline ring to bind to neuromelanin, and antibodies such as an antibody capable of binding to lactotransferrin receptors pathologically expressed on the vasculature in close anatomical proximity to the mesencephalon. Using such lactotransferrin antibodies covalently attached to the active ingredient, preferably covalently attached to chloroquine, chloroquine phosphate, or hydroxychloroquine, competitively inhibits the incorporation of iron into the neurons, and thereby attenuates the pathological incorporation of iron which has been characterized as being contributory to oxidation stress and subsequent neural degeneration in Parkinson's.

Retinal and peripheral membrane protective agents (also called “protectors”) are desirable when the active ingredient is administered long-term, e.g., for a year or more. Chloroquine and related compounds tend to bind to membranes and cause rigidity in the membranes, especially mitochondrial membranes. CQ is a calcium ion ATPase pump inhibitor. In the retina, CQ binds to pigment and produces retinal degeneration. Peripheral membrane protective agents also act to counteract peripheral sympathetic nerve damage occurring in Parkinson's disease, by means of their membrane-stabilizing and neural protective activities.

The compositions of this invention also act as effective agents to counteract loss of sympathetic neuron efferents and attenuated norepinephrine by inducing a supersensitivity to endogenously lower the response threshold to effectors governed by norepinephrine sympathetic neuron fibers.

Preferred retinal and peripheral membrane protectors are selected from the group consisting of calcium citrate, calcium gluconate, calcium lactate, and calcium phosphate, but not calcium carbonate. Preferably the retinal and peripheral membrane protector includes Vitamin D to facilitate gastrointestinal absorption of the calcium. Calcium ions have a high affinity to retinal melanin, accumulate in the eye pigment and competitively inhibit CQ binding to retinal melanin. Also, increasing calcium ion concentration can help restore flexibility to other membranes, especially mitochondrial membranes. Since calcium ions compete with CQ for binding membrane and melanin binding sites, it is preferred that the calcium ions be administered along with the active ingredient in a time-release formulation wherein the calcium ions are released about two to three hours prior to CQ release.

Peripheral metabolism inhibitors are compounds that inhibit breakdown of active ingredients into their metabolites (e.g., for chloroquine and its enantiomers, monodesethylchloroquine and desethylchloroquine and their enantiomers), and thereby increase the active ingredient availability for crossing the blood-brain barrier where it is active for the therapeutic purposes of this invention. CQ is generally more lipophilic than its metabolites, and thus more easily crosses the blood-brain barrier. Use of peripheral metabolism inhibitors can allow dosage reduction of the active ingredient by allowing greater active ingredient incorporation into the central nervous system, and therefore, better Parkinson's treatment efficacy. For example, CQ dosages can be reduced to as low as about 100 mg to 200 mg base equivalents daily.

Peripheral metabolism inhibitors may also serve as retinal protective agents since CQ metabolites more readily bind to eye pigment than CQ itself, and reducing the amount of available metabolites will reduce the amount of retinal degeneration.

The use of such peripheral metabolism inhibitors also helps lower incidence of cardiac and dermatological adverse events associated with CQ metabolites, thereby improving the safety and toxicology profiles of compositions described herein, especially when compared with standard antimalarial agents.

Preferred peripheral metabolism inhibitors are cytochrome P450 2D6 and/or 3A enzyme inhibitors. These can reduce the amount of CQ metabolites present. These inhibitors do not prevent absorption of dopamine, L-dopa or other dopamine agonists, but may interfere with bioavailability of other medications a patient may be taking, and if so, it is preferred that the compositions of this invention containing such P450 inhibitors be administered in the evening, or at another time when the medications they interfere with will not be administered.

Preferred cytochrome (CYP) 2D6 enzyme inhibitors are those selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, terbinafine, racemic mixtures and enantiomers, and suitable pharmaceutical salts of the foregoing.

Preferred daily dosage amounts of the foregoing cytochrome (CYP) 2D6 enzyme inhibitors are as follows:

Amiodarone: about 400 mg to about 800 mg. This is a preferred adjuvant because it acts as an inhibitor upon both CYP 2D6 and CYP 3A.

Celecoxib: about 200 mg to about 400 mg. This adjuvant is known as an antiarthritic agent, and is preferred for use when treating patients having concurrent arthritis.

Chloropheniramine: about 6 mg to about 10 mg. This adjuvant is a histamine H₁ receptor antagonist as discussed below.

Cimetidine: about 400 mg to about 600 mg. Cimetidine is a well-known anti-ulcer and anti-acid reflux agent and is preferably used when treating patients having concurrent gastrointestinal problems, or gastrointestinal problems caused by administration of CQ or other active ingredient. Cimetidine has been used in clinical studies of compositions of this invention with good results, and is a preferable adjuvant due to its absence of adverse cardiac and hypotensive effects.

Clomipramine: about 25 mg to about 100 mg. This is an antidepressant and is preferred when treating patients having concurrent clinical depression.

Fluoxetine: about 20 mg to about 60 mg. This is also an antidepressant and preferred when treating patients having clinical depression.

Levomepromazine: about 15 mg to about 35 mg.

Metoclopramide: about 25 mg to about 30 mg. Like cimetidine, this is an anti-ulcer and anti-acid reflux agent and is preferably used when treating patients having concurrent gastrointestinal problems, or gastrointestinal problems caused by administration of CQ or other active ingredient.

Mibefradil: about 25 mg to about 50 mg.

Moclobemide: about 200 mg to about 30 mg. Moclobemide is an antidepressant, preferably used for treating patients with concurrent clinical depression.

Paroxetine: about 20 mg to about 40 mg. Paroxetine is also an antidepressant, preferably used for treating patients with concurrent clinical depression.

Quinidine: about 200 mg to about 400 mg.

Ranitidine: about 200 mg to about 300 mg. Ranitidine is an anti-ulcer and anti-acid reflux agent and is preferably used when treating patients having concurrent gastrointestinal problems, or gastrointestinal problems caused by administration of CQ or other active ingredient.

Ritonavir: about 600 mg to about 1200 mg.

Sertraline: about 25 mg to about 50 mg. Sertraline is an antidepressant, preferably used for treating patients with concurrent clinical depression.

Terbinafine: about 200 mg to about 400 mg.

Preferred cytochrome P450 3A enzyme inhibitors are those selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, norfluoxetine, racemic mixtures and enantiomers, and suitable pharmaceutical salts of the foregoing.

Preferred daily dosage amounts of the foregoing cytochrome (CYP) 3A enzyme inhibitors are as follows:

Amiodarone: about 400 mg to about 800 mg. This is a preferred adjuvant because it acts as an inhibitor upon both CYP 2D6 and CYP 3A.

Delaviridine: about 400 mg to about 1200 mg.

Indinavir: about 600 mg to about 1200 mg.

Nelfinavir: about 600 mg to about 1200 mg.

Saquinavir: about 1000 mg to about 2000 mg.

Amiodarone: about 400 mg to about 800 mg.

Cimetidine: about 400 mg to about 600 mg.

Ciprofloxacin: about 200 mg to about 200 mg.

Clarithromycin: about 200 mg to about 400 mg.

Diethyl-dithiocarbamate: about 10 mg to about 1000 mg. This compound (carbamic acid) is a metal ion-chelating agent currently being tested for its ability to slow progression of AIDS. As a chelating agent capable of binding iron ions, this is a highly preferred agent for use as a CYP 3A inhibitor.

Diliazem: about 5 mg to about 15 mg.

Erythromycin: about 500 mg to about 1000 mg.

Fluconazole: about 200 mg to about 400 mg.

Fluvoxamine: about 50 mg to about 100 mg. Fluvoxamine is an antidepressant, preferably used for treating patients with concurrent clinical depression.

Itraconazole: about 200 mg to about 400 mg.

Ketoconazole: about 200 mg to about 400 mg.

Mifepristone: about 25 mg to about 50 mg.

Nefazodone: about 50 mg to about 150 mg. Nefazodone is an antidepressant, preferably used for treating patients with concurrent clinical depression.

Mifepristone: about 2000 mg to about 3000 mg.

Norfloxacin: about 250 mg to about 500 mg.

Norfluoxetine: about 40 mg to about 100 mg. Norfluoxetine, like fluoxetine, is an antidepressant, preferably used for treating patients with concurrent clinical depression. Most preferably, a combination of fluoxetine and norfluoxetine is used in formulas for severely depressed Parkinson's patients.

Preferably, the peripheral metabolism inhibitors are administered along with the active ingredients of this invention in the form of a time-release preparation wherein the inhibitors are released about one and a half to about two hours after the retinal and peripheral protective agents, and about one hour before the active ingredient to maximize gastrointestinal absorption and enhance pharmacodynamic interactions.

Enhancing agents are agents, which act to increase levels of active ingredient in the brain or to increase dopamine levels in the brain. Preferred enhancing agents are histamine (H₁) receptor antagonists. These act to counteract increased histamine bioavailability resulting from active ingredient, especially CQ, inhibition of histamine methyltransferase (HMT) and diamine oxidase (DAO, the two primary degradative histamine pathways by chloroquine, and to minimize histamine-associated adverse events, which have been observed with antimalarial treatment formulas.

Although studies have shown that CQ is able to inhibit the action of histamine at certain receptors (i.e., in asthma studies, bronchial arterioles), one of the major problems seen in the treatment of malaria with CQ is pruritis. Pruritis is a histamine-invoked dermatological problem that occurs in about 35% of people being treated for malaria with CQ. It is easily treated with H₁ antagonists like chlorpheniramine or Benadryl, so it is clear that CQ does not inhibit this receptor type peripherally. It is best to treat pruritis by administering an antihistamine before administering CQ, especially when treating patients who are very sensitive to CQ metabolites that, in addition to cardiac side effects, contribute more to the generation of pruritis than parent CQ molecules.

One embodiment of this invention utilizes first-generation histamine H₁ receptor antagonists as adjuvants. First-generation histamine H₁ receptor antagonists are those that are capable of crossing the blood-brain barrier. These agents can cause drowsiness. Such first-generation histamine H₁ receptor antagonists are preferably selected from the group consisting of carbinoxamine maleate, clemastine, diphenhydramine, dimenhydrinate, pyrilamine maleate, tripelennamine, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, and racemic mixtures and enantiomers and suitable pharmaceutical salts of the therapeutic moieties of the foregoing. Other pharmaceutically effective salts of the foregoing compounds than those mentioned above are also useful.

Preferred daily dosages for the foregoing first-generation histamine H₁ receptor antagonists are as follows:

Carbinoxamine maleate: about 10 mg to about 8 mg.

Clemastine: about 3 mg to about 6 mg.

Diphenhydramine: about 50 mg to about 100 mg.

Dimenhydrinate: about 100 mg to about 200 mg.

Pyrilamine maleate: about 100 mg to about 200 mg.

Tripelennamine: about 100 mg—preferably in sustained release form.

Chlorpheniramine maleate: about 12 mg, preferably in sustained release form. Other chlorpheniramine salts may also be used. Preferably, the chlorpheniramine is administered in the form of d-chlorpheniramine, as this form has higher efficacy than the l-form or the racemic form, and use of a more effective form can save space in a capsule in which the composition is packaged.

Brompheniramine maleate: about 12 mg sustained release.

Hydroxyzine hydrochloride: about 50 mg to about 100 mg.

Hydroxyzine pamoate: about 50 mg to about 100 mg.

Cyclizine lactate: about 50 mg to about 100 mg.

Mecllizine hydrochloride: about 40 mg to about 60 mg.

Promethazine hydrochloride: about 50 mg to about 100 mg.

In another embodiment of this invention, a second-generation histamine (H₁) receptor antagonist is used as an adjuvant. Second-generation histamine (H₁) receptor antagonists are not capable of crossing the blood-brain barrier, and therefore do not cause drowsiness. Preferred second-generation histamine (H₁) receptor antagonists are those that do not cause adverse cardiac effects, e.g., torsaides des pointes and arrhythmias.

Preferred second-generation histamine (H₁) receptor antagonists for use as adjuvants herein are selected from the group consisting of acrivastine, cetirizine hydrochloride, astemizole, loratadine and terfenadine, racemic mixtures and enantiomers thereof, and acceptable pharmaceutical salts of the therapeutic moieties of the foregoing.

Preferred daily dosages for second-generation histamine (H₁) receptor antagonists are as follows:

Acrivastine: about 15 mg to about 25 mg.

Cetirizine hydrochloride: about 10 mg to about 20 mg.

Astemizole: about 10 mg.

Loratadine: about 5 mg to about 10 mg.

Terfenadine: about 60 mg.

Compositions of this invention comprising enhancing agents may be prepared in the form of time-release preparations. Preferably the enhancing agent is released concurrently with the active ingredient.

Compositions comprising enhancing agents are capable of affording neuroprotection and can prevent manifestation of Parkinson's disease motor symptoms if treatment is started early in the disease state, i.e., before about fifty percent of the dopamine neurons in the substantia nigra have been lost. Slowing of progression of Parkinson's disease is accomplished by the active ingredient being able to counteract the majority of pathological indices described as contributing to the neurodegeneration seen in Parkinson's Disease.

The foregoing compositions comprising enhancing agents are synergistic with other available Parkinson's disease medications and are capable of prolonging the utility and efficacy of other available Parkinson's disease medications by allowing patients to postpone taking L-Dopa and other available Parkinson's disease medications, allowing for dramatic dose reductions in concomitant Parkinson's disease medications when patients begin taking the compositions of this invention, and by slowing and/or arresting dopamine cell loss, making it no longer necessary to steadily increase dosages of currently-available Parkinson's disease medications.

Compositions of this invention may also comprise an effective amount of at least one adjuvant selected from the group consisting of antioxidants, other retinal protective agents, other neural protective compounds, dopamine or dopamine agonists, and free radical deactivators.

The antioxidant may be any antioxidant known to the art to prevent free radical formation and oxidative degradation of tissues and is preferably selected from the group consisting of probucol, pycnogenol, Vitamin C, Vitamin E, superoxide dismutase, preferably synthetic, BHT, BHA, and melatonin.

The retinal protective agent is a composition administered locally to prevent binding of retinal melanin with CQ, as is known to the art, e.g., alkanes and alcohols of C₁-C₄, ginko biloba and the calcium compounds and vitamin D adjuvants discussed above.

The neural protective compound is any compound known to the art and preferably is selected from the group consisting of selegiline hydrochloride and other monoamine oxidase inhibitors.

The dopamine agonist is any compound known to the art as an anti-Parkinson's treatment and preferably is selected from the group consisting of L-DOPA, pramipexole, ropinerole, bromocriptine, tolcapone, and carbidopa.

The free radical deactivator is any compound known to the art and preferably is selected from the group consisting of superoxide dismutase, selegiline, hydrochloride, and tolcapone.

The compositions of this invention are capable of augmenting dopamine availability, as seen in behavioral results generated in clinical studies thereof, by way of two primary mechanisms: First, CQ inhibits re-uptake of catecholamines including dopamine; and secondly, our analysis reveals that CQ is structurally compatible and pharmacokinetically related to two mixed monamine oxidase A and B inhibitors, namely hydralazine hydrochloride (CAS No. 304-20-1) and quinacrine dihydrochloride (CAS No. 69-05-6), and thus can inhibit degradation of catecholamines.

In one embodiment, a single adult dosage amount of said composition effective for increasing cellular respiration of melanized catecholamine neurons is provided. Active ingredients may be provided in dosages as high as will be tolerated, e.g., malarial dosages up to 500 mg per day, but preferably less than an antimalarial single adult dosage amounts are used, more preferably less than about 1 mM base equivalents, and most preferably less than about 0.5 mM base equivalents of CQ. As is known to the art, the term “base equivalents” refers to amount of active ingredient (e.g., in reference to chloroquine phosphate, refers to the chloroquine minus the phosphate and filler components). A single adult dosage amount with respect to use for alleviation, preventing or stopping progression of symptoms of Parkinson's disease or for other uses described herein will be an amount effective when administered daily to provide the stated therapeutic effect. Compositions of this invention comprising adjuvants that increase the bioavailability of the active ingredient may be administered in active-ingredient dosages as low as about 100 mg to about 200 mg daily.

This invention also provides kits comprising in close proximity, such as in a container or blister pack, effective dosage amounts and forms of the compositions of this invention for single doses, or doses per week, or other appropriate time period, preferably in combination with an adjuvant, such as a peripheral retinal or membrane protective agent, a peripheral metabolism inhibitor, an enhancing agent, an antioxidant, dopamine or dopamine agonist, free radical deactivation, or other adjuvant as discussed above suitable for co-administration with said composition, in effective dosage forms and amounts.

Suitable pharmaceutical carriers are known to the art and include carriers aiding in transport across the blood/brain barrier, such as nanoparticles onto which the compositions are absorbed, coated with a detergent, e.g., as described in Begley, D. J. (1996) “The blood-brain barrier: principles for targeting peptides and drugs to the central nervous system,” J. Pharm. Pharmacol. 48(2):136-46, incorporated herein by reference to the extent not inconsistent herewith.

This invention also provides methods for increasing cellular respiration of melanized catecholamine neurons, and methods for alleviating symptoms or stopping appearance and/or progression of symptoms of Parkinson's and related diseases, and methods for preventing symptoms of on-off syndrome associated with treatment with dopamine or a dopamine agonist, of a patient suffering symptoms of a disease selected from the group consisting of idiopathic and atypical Parkinson's disease, conditions characterized by nigrostriatal degeneration, multiple system atrophy, and vascular Parkinson's disease, as well as non-L-dopa-responsive atypical Parkinsonian disorders, sometimes called “Parkinson's plus syndrome.” Said methods comprise administering to said patient an effective amount of an above composition of this invention. The methods are suitable for any mammal having such melanized neurons or symptoms of Parkinson's disease. Methods for treating or preventing symptoms of Parkinson's Disease and related conditions as described above also comprise identifying patients having such symptoms or at risk of developing them.

Clinical studies have shown that compositions of this invention can effectively improve cognition, alleviate motor symptoms and attenuate the progression of Parkinson's disease and the foregoing related disorders when administered in dosages similar to dosages that are required to treat idiopathic Parkinson's disease.

Further provided herein are methods of making pharmaceutical compositions which are effective for increasing cellular respiration of melanized catecholamine neurons comprising: providing a composition of this invention as described above comprising an active ingredient and an adjuvant, providing a suitable pharmaceutical carrier; and mixing said composition and pharmaceutical carrier to form a composition effective to increase cellular respiration of melanized catecholamine neurons.

Instead of mixing (+)-CQ with (−)-CQ, the method of making the compositions of this invention comprising (−)-CQ may be practiced by starting with racemic chloroquine and removing an amount of (+)-CQ to leave a CQ composition effective to increase cellular respiration of melanized catecholamine neurons.

### DETAILED DESCRIPTION

CQ is capable of protecting neurons against numerous noxious assaults, including systemically administered MPTP (N-methyl-4-phenyl-1,2,3,6-tetrahydropyridine) and intra-nigral injections of MPP+ (1-methyl-4-phenyl-pyridine) its active metabolite, responsible for the generation of PD symptoms humans and animals. Neuroprotection has a definitive role in the prevention of dyskinesias. Therefore, it was altogether gratifying to discover that during the course of our twelve-week pilot trial our CQ formula's (i.e., CQ plus a brain targeting agent) anti-dyskinetic effects appeared to be as poignant as its neuroprotective effects.

Like many neurotherapeutic candidates, CQ's peripheral effects, especially those exerted by the desethylated metabolites, are harmful enough to preclude long-term administration of CQ as is needed to treat the disorders described herein. Additionally, preserving CQ's structural integrity is essential for CQ delivery over the blood brain barrier (BBB) to allow CQ to exert maximal therapeutic benefits in the CNS. By resolving out the S(+)CQ enantiomer, which contributes less to CQ's therapeutic effects and more to the formation metabolites, the long-term safety and tolerability of CQ has been improved. Furthermore, by combining R(−)CQ with time-released brain targeting agents and enhancing agents, which maximize CQ absorption and delivery over the blood-brain barrier (BBB), clinically significant effects can be achieved while administering less than one half of the approved amount of the racemic mixture.

Neuroprotection has a definitive role in the prevention of dyskinesias. During the course of our twelve-week pilot trial, it was discovered our simplest CQ formula (i.e., time release CYP2D6/3A inhibitor+CQ) alleviated PD symptoms, ameliorated levodopa-induced dyskinesias (LIDs)/motor fluctuations (MFs) and allowed for significant reductions to be implemented in concomitant PD medications as shown in Example 2.

Drug-induced movement disorders (DID) pose a serious problem to physicians, complicating the successful effective pharmacotherapeutic treatment of patients diagnosed with Parkinson's Disease (PD), schizophrenia and other dopamine (DA)-associated disorders. During the course of levodopa (L-dopa) therapy the majority of patients will go on to develop MFs and/or LIDs. MFs frequently occur in persons having early onset Parkinson's Disease (PD); whereas, LIDs commonly manifest early on in cases of severe PD. Neuroleptic Malignant Syndrome (NMS), which is often mistakenly associated with schizophrenia and neuroleptic medications, also effects PD patients, manifesting most frequently following dopaminergic dose reductions, drug withdrawal, and in sensitive patients, during dopaminergic “wearing-off” periods between dosages. Additionally, it has recently been established that 15%-30% of patients undergoing embryonic putamen cell transplants develop “run away” dyskinesias even in the absence of L-dopa or dopaminergic drug therapy.

Tardive Dyskinesia and NMS are promoted by “traditional” antipsychotic medications including phenothiazines and decanoates, such as haloperidol and fluphenazine which are dopamine antagonists. Traditional antipsychotic medications are just as, if not more, effective in alleviating psychosis than the newer more expensive atypical medications. However, these older medications often produce more side effects and have a higher dyskinetic profile. Tardive dyskinesia begins to manifest in 20-25% of patients within 5 years of being initiated on DA antagonists.

Hyperkinetic and hypokinetic disorders share a certain similarity in neuropathic characteristics in that they all involve abnormal output from the basal ganglia. TD and LID are considered to be hyperkinetic disorders. Surgical procedures such as bilateral posteroventral pallidotomy and thalamotomy, have been used to treat non-refractory TD and disabling LID in patients having schizophrenia and PD, respectively. Parkinsonism and extrapyramidal side effects (EPS), both of which can be induced by neuroleptic medications, are considered hypokinetic disorders. These symptoms can be alleviated by increasing DA medications in PD or reducing the amount of DA antagonizing medication being taken by persons with schizophrenia.

The use of L-dopa and typical neuroleptics is limited by the expression of these secondary movement disorders. CQ is able to counteract these degenerative mechanisms. CQ's ability to ameliorate the DIDs, as was seen in our pilot trial, and CQ's ability to counteract the pathological processes underlying their formation makes CQ a highly desirable drug to use in conjunction with these agents. CQ given in combination and/or concomitantly with these therapeutics reduces their DID profiles and prolongs their efficacy and utility for successfully treating these DA-related disorders long-term. Additionally, LIDs are known to resolve following dose reductions of L-dopa and/or high dyskinetic profile DA agonists. Supplementing PD pharmacotherapy with agents that behave synergistically with L-dopa and/or permit dosage reductions to be implemented in conventional PD medications, as occurred in the patients experiencing LIDs prior to enrollment in our clinical trial, serves to further ameliorate existing LIDs.

CQ is a potent inhibitor of NF-kappaB degradative molecule, IF-kappaBα, whereby it maintains the activation of NF-kappaB anti-apoptotic transcriptional and brain/glial-derived neurotrophic factors capable of slowing and/or arresting the neurodegeneration seen in PD, TD and negative forms of schizophrenia. Also, (R)CQ binds selectively to guanine-containing base pairs in DNA making it resistant to oxidative damage, DNAase and fragmentation, thereby reducing the incidence of cellular apoptosis. Additionally, with oxidative stress being a problem in PD and contributory to the development of TD and negative symptoms of schizophrenia, (R)CQ is most effective for preventing oxidative damage precipitated by neuromelanin (NM), free iron, cellular oxidants and other free radicals that contribute to formation and progression of these illnesses. The use of a P450 enzyme inhibitor augments neuroleptic bioavailability of CQ, which is conducive to attenuating TD symptoms. In addition, the use of a P450 inhibitor as a brain-targeting agent allows for dose reductions to be implemented to neuroleptic medications without the drawback of perpetuating the manifestation of TDs.

The opiate peptides implicated in DIDs (i.e., both LIDs and TD) are enkephalin (ENK) contained in medium-sized D2 receptor-bearing GABA neurons in the indirect striatopallidal pathway and dynorphin (DYN)/substance P (Sub P) co-expressed in the medium aspiny GABA-producing D1 receptor-bearing neurons in the direct striatonigral pathway. ENK delta (δ) and mu (μ) are opiate receptor agonists; whereas, DYN binds to δ and μ, but most specifically to kappa (κ) receptors. CQ is a potent κ and μ opiate receptor agonist. Additionally, CQ is a “target based” therapeutic agent, capable of accumulating within the striatum, substantia nigra (SN), thalamus, mesencephalon, brain stem and cerebellum in a combined concentration ratio of 99% to 1%, as opposed to the cerebral cortex. Most agents exert global, rather than targeted or anatomically-specific, effects. This renders many agents, which are highly effective at attenuating dyskinesias when administered locally, impractical or even harmful when administered systemically.

As stated above, CQ is a potent κ and μ opiate receptor agonist. Thus far, efforts to minimize DIDs by manipulating the opiate receptors in humans have been limited to the use of naltrexone and naloxone, both non-specific opiate receptor antagonists. Perhaps this is because agonism of kappa receptors residing deep within the cerebral cortex promotes sedation, ataxia and decreased locomotion. However, CQ does not accumulate in the cerebral cortex and therefore cannot agonize cortical κ and μ receptors.

In PD, increased κ expression is found in areas of high excitatory amino acid (EEA) (i.e., glutamate) transmission, such as the striatum and the globus pallidus internus (GPi). κ agonism in the GPi reduces the release of glutamate from subthalamic afferents, thereby diminishing hypokinetic disorders, PD akinesia/motor symptoms and EPS. Also, κ agonists increase locomotor activity in monoamine-depleted mammals, independently and synergistically with L-dopa/DA agonists. Therefore, CQ effectively reduces the threshold dose that is required for L-dopa/DA agonists to evoke a similar response. Lowering the dose of DA L-Dopa agonists effectively ameliorates LIDs.

Striatal and basal ganglia κ and μ receptors are effectively agonized by CQ when administered in conjunction with brain targeting/enhancing agents in the dosages described herein. This serves to enhance L-dopa/DA agonist efficacy at lower therapeutic dosages, thereby prolonging the utility of these agents for treating PD motor symptoms. Lowering the threshold dose of L-dopa/DA agonists by combining these agents with CQ, diminishes LIDs. CQ's ability to attenuate EEA transmission in the GPi via kappa-induced inhibition of synaptic glutamate release diminishes hypokinetic disorders, such as PD and EPS.

CQ exerts dopaminergic effects and acts as a mild DA neural reuptake inhibitor. DA reuptake inhibitors prolong the synaptic bioavailability of endogenous DA, which is useful for reducing the symptoms of PD, EPS, LIDs, and TD and treating the negative symptoms of schizophrenia. Most DA reuptake inhibitors exert anti-dyskinetic effects; but, due to their side effects, they are often difficult for patients to tolerate. Our clinical results established that CQ promotes PD symptom relief and generates a significant anti-dyskinetic effect without compromising patient well-being and/or diminishing L-dopa efficacy. Additionally, CQ inhibits DA receptor recycling via its lysosomotropic action without impeding DA receptor internalization. Thus, CQ diminishes DA receptor “sensitization” as a result of treatment with L-dopa.

In PD, glutamate (an excitatory amino acid [EEA]) stimulation of NMDA receptors in the corticostriatal and basal ganglia is hyperactive in PD, which leads to neurodegeneration and motor dysfunction. The use of glutamate antagonists is limited by the side effects that non-specific “global” NMDA antagonism produces. These side effects include psychiatric disturbance, ataxia, dissociative anesthesia and diminishment of L-dopa efficacy. A more effective method for inhibiting NMDA receptor activity is to inactivate the NMDA NR2A and NR2B subunits with chloroquine, specifically in the striatum and basal ganglia.

Chloroquine inactivation of the NR2A and NR2B subunits alleviates hypokinetic systems of PD and hyperkinetic DIDs, such as LIDs and TD. The neuroprotection that is achieved by CQ inactivation of NR2A and NR2B impedes the progression of PD and interrupts the degenerative mechanisms underlying LIDs, TDs and the negative symptoms of schizophrenia.

CQ inhibits acetylcholinesterase (AChE) and butyrylcholinesterase (BCHE) and is an ACh muscarinic and nicotinic antagonist. R-CQ is most effective for inactivating ACh receptors. Anti-cholinergic (muscarinic) drugs improve PD symptoms up to 20% and are frequently prescribed to treat EPS. Additionally, cholinergic hyper-excitation contributes to neurodegeneration in PD and schizophrenia.

Additionally, overactive ACh and glutamate efferents to the substantia nigra zona compacta (SNc) underlie the DA cell loss seen in both PD and Progressive Supranuclear Palsy (PSP). Suppressing ACh and glutamate stimulation of DA neurons in SNc, CQ slows or prevents the progression of SNc DA cell loss and the manifestation of motor deficits seen in both PD and PSP.

The term “increasing cellular respiration” means measurably increasing oxygen consumption, increasing aerobic cellular respiration and reducing anaerobic cellular respiration, e.g., as measured by lactate in the cerebral spinal fluid.

The term “diminishing oxidative degradation of dopamine neurons in the substantia nigra and basal ganglion” means measurably diminishing such degradation as measured by assays known to the art, including measures of free iron ion availability, lipid peroxidation by-products such as malondialdehyde formation, and oxygenated radical formation.

The term “alleviating symptoms of Parkinson's disease or related conditions” means measurably reducing, inhibiting, attenuating and/or compensating for at least one symptom of Parkinson's disease or related condition, such as tremor, postural imbalance, rigidity, bradykinesia, akinesia, gait disorders, and on/off fluctuations. These symptoms may result from toxic metabolite formation during neuromelanin (NM) synthesis, heightened affinity of endogenous and exogenous toxins for NM, mitochondrial impairment, increased oxidative stress potentiated by reduced levels of antioxidants, protein oxidation and lipid peroxidation, augmented iron content and abnormal Fe(II)/Fe(III) ratios, and the accumulation of extracellular protein peptide fragments, which conditions may also be alleviated by the compositions of this invention.

The compositions of this invention containing (−)-CQ should have more (−)-CQ or CQ mixed, complexed, or covalently linked with an adjuvant than (+)-CQ because the toxic metabolites of (+)-CQ make it less suitable for long-term use, and the better melanin-binding properties of (−)-CQ, its longer half life and lower clearance make it more effective for long-term administration (e.g., at least about six weeks, more preferably, about two years, and most preferably, at least about ten years or more).

An effective amount of the compositions of this invention is an amount necessary to produce a measurable effect. For example, an effective amount of the compositions of this invention to increase cellular respiration measurably increases cellular respiration by assays known to the art as discussed above. In compositions containing (−)-CQ, the effect may be produced by the (−)-CQ, or partially by the (−)CQ and partially by (+) CQ. Similarly, an effective amount of a composition of this invention to alleviate or stop the progression of symptoms of Parkinson's Disease is an amount which does so based on art-known tests such as the Unified Parkinson's Disease Rating Scale and the Tinetti Gait and Balance Assessment Tool, comparing symptoms of treated patients with symptoms of the same patients prior to and/or after treatment, or with symptoms of untreated patients at the same stage of Parkinson's Disease.

Preventing symptoms of Parkinson's Disease includes identifying patients at risk for developing such symptoms. Identification of patients susceptible to onset of Parkinson's Disease may be done by genetic testing, prediction from family history or other means known to the art such as PET scans. When symptoms of Parkinson's do not develop, or do not develop to the expected (average) degree, they are considered to have been prevented by the methods and compositions of this invention.

Preventing on-off symptoms in patients being treated with L-Dopa or like medications means measurably stopping or decreasing such symptoms as compared with patients at similar stages of Parkinson's Disease being treated with such medications.

The compounds of this invention may be formulated neat or may be combined with one or more pharmaceutically acceptable carriers for administration, such as solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular mixture employed, the mode of administration and the severity of the condition being treated. However, in general, satisfactory results are obtained when the active ingredients of the invention are administered at a daily adult dosage of from about 0.5 to about 1000 mg, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation. Preferably a single daily adult dose comprises less than about 1 mM, and more preferably less than about 0.5 mM base equivalents, more preferably less than about 1 mM, and more preferably less than about 0.5 mM base equivalents. Active-ingredient dosages of between about 100 mg and about 200 mg base equivalents daily may be used, especially in combination with adjuvants which increase bioavailability of the active ingredient as described above.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compositions is preferred. Time-release formulas as described above are desirable in many cases as taught herein. In some cases it may be desirable to administer the compounds to the patient's airways in the form of an aerosol.

The compounds of this invention may also be administered parenterally or intraperitoneally. Solutions or suspensions of these active compounds as a freebase or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

“Suitable pharmaceutical carriers” as referred to herein include distilled and pharmaceutical grade water, but do not include water or buffers unsuitable for administration to a human patient.

There are several mechanisms by which neuromelanin may contribute to symptoms of Parkinson's disease by contributing to formation of toxic products including superoxide and hydroxy radicals, which catalyze lipid peroxidation, and oxidation of NADH resulting in disruption of the neuron's respiration and reducing the amount of energy available to the neurons via aerobic respiration.

Neuromelanin can be considered a waste product of catecholamine degradation and gradually accumulates within the cytosol of catecholamine neurons throughout one's lifetime. Dopamine is autoxidized to cytotoxic and reactive oxygenated species such as 6-hydroxydopamine (6-OHDA) and semiquinone radicals. Low glutathione levels contribute to oxidative stress in Parkinson's disease, and allow available hydrogen peroxide to be further catalyzed by iron into highly toxic superoxide radicals and hydroxyl radical species as well as semiquinone radicals. Dopamine and L-DOPA interaction with superoxide radicals augments depletion of glutathione, leading to a downward spiral of detrimental reactions.

Monoamine oxidase forms toxic metabolites from a number of substances such as beta-carboline derivatives and tetrahydroisoquinoline that are present in excessive amounts in the cerebral spinal fluid of people with Parkinson's Disease. These toxic metabolites have high affinity to neuromelanin, and once bound may cause almost complete arrest of ATP production, resulting in impaired respiration, loss of energy available to the neurons and massive melanized cell loss which leads to symptoms of Parkinson's Disease. Inhibitors of monoamine oxidase B such as Deprenyl prevent formation of these toxic metabolites. Iron also tends to bind to neuromelanin, resulting in a cascade of pathogenic reactions leading to neuronal death. Increasing iron concentrations in basal ganglia are observed with normal aging, and in patients with Parkinson's Disease, iron is pathologically elevated with high ferric/ferrous ion ratios. The ferric ions contribute, with 6-OHDA, to the formation of harmful superoxide and hydroxyl radicals leading to lipid peroxidation and cell breakdown.

Iron chelators have been shown to reverse impaired mitochondrial respiration caused by 6-OHDA inhibition of NADH dehydrogenase. 6-OHDA catalyzes the release of iron from intracellular ferritin stores which in turn catalyzes lipid peroxidation. This toxic chain of events can be inhibited by superoxide dismutase. Both iron chelators and chloroquine phosphate have been found to limit the availability of free iron, so that it is not available to catalyze these toxic reactions.

The iron transporter protein, diferric transferrin, which delivers iron throughout the body also contributes to loss of energy available to the neurons by interfering with availability of reduced NADH. Chloroquine phosphate has been found to inhibit intracellular oxidation of NADH by melanin.

Chloroquine phosphate binds to neuromelanin and does not inhibit enzymatic synthesis of iron into biologically essential compounds. It not only prevents incorporation of iron into neurons, but also inhibits the release of iron from intracellular iron pools. In addition chloroquine phosphate has been found to heighten an astrocytic immune response against accumulation of extracellular protein deposits in the brain contributing to Alzheimer's Disease.

The (−) isomer of chloroquine is an even more effective neuromelanin binder than racemic chloroquine because it breaks down less peripherally, has a longer half-life and lower clearance, and so is more available to cross the blood brain barrier, as well as having a stabilizing effect on DNA. It is therefore preferred for use in this invention.

### EXAMPLES

### Example 1

Enantiomers of chloroquine phosphate were isolated according to the procedure of Stalcup, A. M. et al. (1996), Analytical Chemistry 68:2248-50. Comparisons of these enantiomers with respect to ability to inhibit diamine oxidase and bind to neuromelanin are performed in vitro. Results show significantly enhanced ability of the active enantiomer in both assays to inhibit diamine oxidase and bind neuromelanin.

### Example 2

A within-subjects, open labeled, pilot study was performed to evaluate the safety and tolerability CQ and enantiomeric CQ (test compounds) for the treatment of motor disorders in adults having a diagnosis of Idiopathic Parkinson's Disease (IPD) and Symptomatic Parkinson's Disorders. Functional “on” and “off” evaluations were administered using the Unified Parkinson's Disease Rating Scale and timed tapping tests for assessment pre-treatment, during treatment, and two weeks post-treatment changes in well-being. The treatment period assessed the safety and durability of response for up to eight weeks. An initial two-week pre-treatment period established each participant's baseline neurophysiological and well-being measures. A final evaluation, administered following a two-week treatment withdrawal period, evaluated each participant for symptom restoration.

Ten adults between 30-75 years of age, having a confirmed diagnosis of stage I-III PD, received a reverse titration of the test medication during the first week of treatment. This is followed by a one-time-per-day maintenance dose of 155 mg per day taken with the evening meal.

During the initial 24-hour treatment period, subjects were instructed to take 155 mg of the test medication four times per day. On study days 2 and 3, subjects were instructed to take 155 mg of test medication 3 times per day. On study days 4, 5 and 6, subjects were instructed to take 155 mg of test medication 2 times per day. On study day 7, subjects were instructed to take 155 mg of the test medication daily with their final meal of the day. On treatment day 10, physicians determined final maintenance dose to be taken each evening with the subject's final meal of the day for the duration of the treatment period. The maintenance dose was kept at 155 mg test medication per day or adjusted to a lower or higher dose, e.g. down to 100 mg if the subject was showing improvement but having gastrointestinal or other discomforts. The dose was increased up to 200 mg or 255 mg per day if the subject had.not experienced symptom relief.

Improvements in pre-treatment (baseline) scores on the above-described or similar measuring instruments, and/or decline in function and score values following the medication withdrawal period were used for assessment. Subjects were checked for the occurrence of adverse events during the study each visit. Laboratory evaluations (chemistry and hematology profiles) were performed at pre-treatment screening and during the treatment period on days 10, 28 and 56, and also during the two-week post-treatment exit evaluation.

Pre-treatment (baseline) measurements were taken during the initial two-week pre-treatment evaluation period. The pre-treatment scores were averaged to determine each patient's baseline neurophysiological and well-being measurements. Two separate neurophysiological and well-being evaluations were administered on treatment days 10 and 56. Medication was discontinued immediately after the neurophysiological and well-being evaluation administered on treatment day 56. Patients were seen for one additional exit interview including complete physical examination and laboratory evaluations two weeks after the experimental treatment was discontinued.

Baseline scores obtained during the two-week pre-treatment period were compared to scores obtained during treatment days 10 and 56 to determine any changes in patient status throughout the treatment period. The final two-week post-treatment evaluation was to determine patient well being and to increase the dosage of patients' concomitant PD medications that were reduced during the study medication period. Improvement of motor symptoms from pre-treatment and medication period were evaluated, as well as improved well-being. Within-subject improvement was analyzed using a t-test of differences for scores from the pre-test condition to the post-test condition using a p value of 0.05. Variables were summarized by treatment group according to subgroups of gender, race, and age.

Changes from pre-treatment evaluations to days 7, 14, 28, 42 and 56 in each clinical sign and symptom were summarized by treatment group. Potentially clinically significant laboratory values and mean changes from baseline of vital signs data were summarized within both treatment groups. Times to resolution/improvement of symptoms after treatment were also summarized. Subject satisfaction data and subject symptoms collected from questionnaires were also summarized by treatment group and analyzed. Based on an adverse event rate of 3%, the treatment group sizes used provided approximately 80% power to detect significance difference at the p value of 0.05 (two-tailed) significance level.

Significant improvement in symptoms and halting of progression of symptoms both during and post-treatment was observed.

Preliminary clinical studies yielded the following results:

### The Use of Chloroquine Diphosphate for Multiple Symptom Atrophy (MSA)

### Protocol # PD/CQ CASE #1 (MSA)

| | | Symptoms | Diagnosed | | | |
|---|---|---|---|---|---|---|
| Birth | | First | by | Present | Height | Weight |
| Year | Sex | Noticed | Physician | Stage PD | (inches) | (pounds) |
| 1935 | M | n/a | 1998 | V | 71 | 230 |

| | BASELINE | Rx DAY 14 | BL-Day |
|---|---|---|---|
| UPDRS | SCORES | SCORES | 14 = change |
| ADL OFF | 34 | 32 | 2 |
| ADL ON | 34 | 32 | 2 |
| MOTOR OFF | 50 | 46 | 4 |
| MOTOR ON | 47 | 44 | 3 |
| ADL + MOTOR OFF | 84 | 78 | 6 |
| ADL + MOTOR ON | 81 | 76 | 5 |
| TOTAL UPDRS | 85 | 82 | 3 |

| TIMED TAPPING | BASELINE | Rx DAY 14 | Day 14-BL |
|---|---|---|---|
| Right Hand “OFF” | 44 | 54 | +10 |
| Right Hand “ON” | 47 | 51 | +4 |
| Left Hand “OFF” | 40 | 48 | +8 |
| Left Hand “ON” | 46 | 49 | +3 |

Case #1 was the first patient to be administered CQ plus brain targeting agent (BTA; cimetidine) having a confirmed diagnosis of Multiple Symptom Atrophy (MSA). Motor improvements can be seen in both the timed tapping and UPDRS scales scores between baseline and treatment day 14. On the medication day 35 visit, patient reported less freezing (i.e.—OFF time) during the previous 10 days and an increase in concentration. The patient's speech therapist and physical therapist, both seen bi-weekly, reported respective improvements in speech and range of motion. However, following the day 35 visit, a violation of the protocol occurred that necessitated the disqualification of this patient from enrollment.

### The Use of Chloroquine Diphosphate for Parkinson's Plus Disorders

### Protocol # PD/CQ CASE #2 (Parkinson's w/Concurrent Dementia)

| | | Symptoms | Diagnosed | | | |
|---|---|---|---|---|---|---|
| Birth | | First | by | Present | Height | Weight |
| Year | Sex | Noticed | Physician | Stage of PD | (inches) | (pounds) |
| 1922 | M | 1996 | 1996 | III | 71.5 | 132 |

| | BASE- | Rx | BL-Day | Rx | BL-Day |
|---|---|---|---|---|---|
| | LINE | DAY 14 | 14 = | DAY 56 | 56 = |
| UPDRS | SCORES | SCORES | change | SCORES | change |
| ADL OFF | 14 | 18 | −4 | 12 | 2 |
| ADL ON | 12 | 15 | −3 | 11 | 1 |
| MOTOR OFF | 28.5 | 41 | −12.5 | 30 | 1.5 |
| MOTOR ON | 22.5 | 27.5 | −5 | 24 | 1.5 |
| ADL + MOTOR | 42.5 | 59 | −16.5 | 42 | 0.5 |
| OFF | | | | | |
| ADL + MOTOR | 34.5 | 42.5 | −8 | 35 | −0.5 |
| ON | | | | | |
| TOTAL UPDRS | 39.5 | 52.5 | −13 | 39 | 0.5 |

| | BASE- | | Day | | Day |
|---|---|---|---|---|---|
| TIMED TAPPING | LINE | Rx DAY 14 | 14-BL | Day 56 | 56-BL |
| Right Hand “OFF” | 47 | 63 | +16 | 58 | +11 |
| Right Hand “ON” | 59 | 56 | −3 | 64 | +5 |
| Left Hand “OFF” | 52 | 57 | +5 | 69 | +17 |
| Left Hand “ON” | 59 | 53 | −6 | 62 | +3 |

Case #2 enrolled having a confirmed diagnosis of stage III Parkinson's Disease with progressive diminishment in cognitive function (Mini-Mental State Exam score of 24, dementia≧24). Similar to Case #1, this patient appeared to have dramatic improvements in cognition and memory while taking CQ+BTA. Two weeks post withdrawal from CQ, both the patient and his wife reported reemerging difficulty in “word finding” and a significant decline in both concentration and memory. The patient requested to be put back on and resumed taking CQ+BTA a week after withdrawal.

Two properties make CQ a likely agent to improve memory and cognitive function. One being that CQ (especially the (+)-CQ enantiomer) is an acetylcholine esterase inhibitor (AChE), which would augment ACh levels (the memory neurotransmitter) in the brain. While this may have contributed to the patient's increase in memory and cognition, ACh is also known to contribute to Parkinson's disease symptomology. As was observed in the timed tapping (tt) and UPDRS evaluations, there appears to be a significant “motor functional” improvement. The second reason for cognitive improvement could be a result of heightened brain tissue oxygenation while the patient was taking CQ. CQ and hydroxychloroquine (HCQ) are both agents known to increase red blood cell (RBC) O₂ absorption and delivery, via mechanisms of a Bohr shift promoted by alterations in physiological pH.

### The Use of Chloroquine Diphosphate for Atypical Parkinson's Disease (APD) Including Reduction of Cognitive Symptoms

### Protocol # CQ/PD CASE #3 (Atypical Parkinsonian Disorder)

| | | | Diagnosed | | | |
|---|---|---|---|---|---|---|
| Birth | | Symptoms | by | Present | Height | Weight |
| Year | Sex | First Noticed | Physician | Stage PD | (inches) | (pounds) |
| 1926 | M | 2001 | April 2001 | II | 69 | 207 |

| | BASE- | Rx | BL-Day | Rx | BL-Day |
|---|---|---|---|---|---|
| | LINE | DAY 14 | 14 = | DAY 56 | 56 = |
| UPDRS | SCORES | SCORES | change | SCORES | change |
| ADL OFF | 12 | 9 | 3 | 9 | 3 |
| ADL ON | 12 | 9 | 3 | 9 | 3 |
| MOTOR OFF | 19 | 17.5 | 1.5 | 13.5 | 5.5 |
| MOTOR ON | 20 | 16.5 | 3.5 | 12 | 8 |
| ADL + MOTOR | 31 | 26.5 | 4.5 | 22.5 | 9.5 |
| OFF | | | | | |
| ADL + MOTOR | 32 | 25.5 | 6.5 | 21 | 11 |
| ON | | | | | |
| TOTAL | 34 | 27.5 | 6.5 | 24 | 10 |
| UPDRS | | | | | |

| TIMED | | Rx | Day | | |
|---|---|---|---|---|---|
| TAPPING | BASELINE | DAY 14 | 14-BL | Day 56 | BL-Day 56 |
| Right Hand | 84 | 80 | −4 | 89 | +5 |
| “OFF” | | | | | |
| Right Hand | 61 | 80 | +19 | 68 | +7 |
| “ON” | | | | | |
| Left Hand | 92 | 77 | −15 | 79 | −13 |
| “OFF” | | | | | |
| Left Hand | 80 | 75 | −5 | 74 | −6 |
| “ON” | | | | | |

Case #3 was diagnosed with Parkinsonism less than one year prior to the study. Review of the outside patient records suggests that this patient had an atypical Parkinson's disorder. In addition, there was no significant levodopa response prior to acceptance into the study. In fact, this patient subsequently failed to follow instructions regarding co-administration of Sinemet with CQ+BTA and stopped all other anti-Parkinson medications for about 10 days without any deterioration in Parkinsonian symptoms. This is a significant indication that this patient does not have the idiopathic form of Parkinson's disease.

Case #3 was administered treatment day 14 UPDRS functional off/on evaluations on Nov. 21, 2001. Patient was doing well even while he had erroneously discontinued taking Sinemet two weeks prior when he began taking CQ+BTA. Patient was instructed to resume taking Sinemet CR 50/100 three times per day and Sinemet 10/100 twice daily. No adverse events or motor improvements were noted during the treatment day 35 visit, but patient reported a sight increase in mental activity. Patient was given an increased dose of CQ from 150 mg to 200 mg per day, following his day 35 visit. By treatment day 56 patient reported that since increasing his dose of CQ, he has “come alive.” He reported increased mental clarity and better mobility, confirmed by physical and occupational therapists. Patient requested to be put back on and resumed taking CQ 200 mg/day+BTA 400 mg/day immediately following his exit evaluation visit. Approximately 15 days after the study, patient reported decrease in stability and asked to resume taking the study drug.

### The Use of Chloroquine Diphosphate to Reduce Dopamine Agonist Dosage

### Protocol #CQ/PD #4 (Parkinson's Disease)

| | | | Diagnosed | | | |
|---|---|---|---|---|---|---|
| Birth | | Symptoms | by | Present | Height | Weight |
| Year | Sex | First Noticed | Physician | State PD | (inches) | (pounds) |
| 1936 | M | 1969 | 1972 | III | 73 | 169 |

| | BASE- | Rx | BL-DAY | Rx | BL-DAY |
|---|---|---|---|---|---|
| | LINE | DAY 14 | 14 = | DAY 56 | 56 = |
| UPDRS | SCORES | SCORES | CHANGE | SCORES | CHANGE |
| ADL OFF | 15 | 15 | 0 | 19 | −4 |
| ADL ON | 13 | 11 | 2 | 16 | 2 |
| MOTOR | 21.5 | 27 | −5.5 | 18.5 | 3 |
| OFF | | | | | |
| MOTOR ON | 18.5 | 10.5 | 8 | 13.5 | 5 |
| ADL + | 36.5 | 42 | −5.5 | 37.5 | −1 |
| MOTOR | | | | | |
| OFF | | | | | |
| ADL + | 31.5 | 21.5 | 10 | 29.5 | 2 |
| MOTOR | | | | | |
| ON | | | | | |
| TOTAL | 40.5 | 28.5 | 12 | 32.5 | 8 |
| UPDRS | | | | | |

| | | Rx | DAY | DAY | DAY |
|---|---|---|---|---|---|
| TIMED TAPPING | BASELINE | DAY 14 | 14-BL | 56 | 56-BL |
| Right Hand “OFF” | 136 | 133 | −3 | 105 | −32 |
| Right Hand “ON” | 104 | 133 | +29 | 145 | +41 |
| Left Hand “OFF” | 111 | 86 | −25 | 102 | −9 |
| Left Hand “ON” | 114 | 102 | −12 | 120 | +6 |

Case #4 continued to maintain him on a lower dose of anti-Parkinson medication after the study. Approximately 20 days after the end of the study, the patient reported a need to increase his anti-Parkinson's medication and asked to continue the study drug. His levodopa-induced dyskinesias were improved. This patient felt that as a result, he was functioning better than before beginning the study medication and reported being almost without dyskinesias.

Two weeks post-treatment, the patient reported feeling well but was experiencing a slight increase in dyskinesias. Once stabilized on chloroquine diphosphate, the patient was able to reduce the dosage of his daily concomitant Parkinson's medications to approximately two-thirds of his pre-study dosage. Approximately 19 days after discontinuing chloroquine diphosphate, this patient expressed a strong desire to resume taking it. This patient's greatest benefit during the medication period was a diminishing of uncontrollable dyskinesias.

The patient's re-emergence of symptoms post-withdrawal coincides with the pharmacological mean residence time (MRT) and half-life of chloroquine diphosphate. As anticipated, chloroquine diphosphate increased cellular respiration and thereby augmented DA synthesis, storage and release in the striatum. We have hypothesized that chloroquine diphosphate would prolong the utility and efficacy of L-Dopa, both by exerting a neuroprotective effect and by its apparent synergy with L-Dopa and dopaminergic agents in promoting dopamine (DA)-associated behavioral effects in animal models. Study results support this hypothesis.

### Protocol #CQ/PD Case #5 (Parkinson's Disease)

| | | | Diagnosed | Present | | |
|---|---|---|---|---|---|---|
| Birth | | Symptoms | by | State | Height | Weight |
| Year | Sex | First Noticed | Physician | PD | (inches) | (pounds) |
| 1927 | F | 1995 | 1995 | III | 65.5 | 144 |

| | BASELINE | Rx DAY 14 | BL-DAY 14 = | Rx DAY 56 | BL-DAY 56 = |
|---|---|---|---|---|---|
| UPDRS | SCORES | SCORES | CHANGE | SCORES | CHANGE |
| ADL OFF | 13 | 11 | 2 | 7 | 6 |
| ADL ON | 11 | 7 | 4 | 5 | 6 |
| MOTOR OFF | 29 | 22.5 | 6.5 | 20.5 | 8.5 |
| MOTOR ON | 13.5 | 14.5 | −1 | 14.5 | −1 |
| ADL + MOTOR OFF | 42 | 33.5 | 8.5 | 27.5 | 14.5 |
| ADL + MOTOR ON | 24.5 | 21.5 | 3 | 19.5 | 5 |
| TOTAL UPDRS | 29.5 | 28.5 | 1 | 26.5 | 3 |

| | | Rx | DAY | DAY | DAY |
|---|---|---|---|---|---|
| TIMED TAPPING | BASELINE | DAY 14 | 14-BL | 56 | 56-BL |
| Right Hand “OFF” | 84 | 112 | +28 | 105 | +21 |
| Right Hand “ON” | 117 | 129 | +12 | 132 | +15 |
| Left Hand “OFF” | 70 | 105 | +34 | 114 | +34 |
| Left Hand “ON” | 112 | 125 | +13 | 118 | +6 |

Case #5's anti-Parkinson medications were appropriately reduced because of an increase in dyskinesias with chloroquine diphosphate administration. The anti-Parkinson medication thereafter was not changed. There was some improvement in UPDRS ADL on and off scores and UPDRS off but not on scores, along with improvements in timed tapping scores, all of which suggest that this patient was significantly improved with administration of chloroquine diphosphate.

This patient appeared to tolerate the study medications very well and seemed to respond significantly even by Day 7 of the medication period. Due to increased dyskinesias, on treatment Day 10 the patient had her dosage of concomitant Sinemet reduced by one-third of the dosage she required at the screening visit. During this patient's “off” medication UPDRS evaluations at Baseline (pretreatment), patient required a wheel chair. On treatment Day 14, patient was able to come in for her “off” medication evaluations using a walker. By treatment Day 35, patient reported having less freezing, no wearing off effects, and being more “even” throughout the day. She was able to get up and use the bathroom at night, which allowed her to discontinue using a commode. However, two weeks after withdrawal from the study medications, the patient had to resort back to using a bedside commode and reported experiencing an increase in instability. She expressed a desire to resume the study medications and did so.

### The Use of Chloroquine Diphosphate to Reduce Cognitive Symptoms of Parkinson's Disease

### Protocol CQ/PD Case #6 (Parkinson's Disease)

| | | Symptoms | | Present | | |
|---|---|---|---|---|---|---|
| Birth | | First | Diagnosed by | State | Height | Weight |
| Year | Sex | Noticed | Physician | PD | (inches) | (pounds) |
| 1943 | F | 1998 | 1999 | II | 65 | 120 |

| | BASELINE | Rx DAY 14 | BL-DAY 14 = | Rx DAY 56 | BL-DAY 56 = |
|---|---|---|---|---|---|
| UPDRS | SCORES | SCORES | CHANGE | SCORES | CHANGE |
| ADL OFF | 9 | 8 | 1 | 7 | 2 |
| ADL ON | 7 | 7 | 0 | 6 | 1 |
| MOTOR OFF | 25 | 23.5 | 1.5 | 15 | 10 |
| MOTOR ON | 16 | 15.5 | .5 | 11 | 5 |
| ADL + MOTOR OFF | 34 | 31.5 | 2.5 | 22 | 12 |
| ADL + MOTOR ON | 23 | 22.5 | .5 | 17 | 6 |
| TOTAL UPDRS | 29 | 27.5 | 1.5 | 21 | 8 |

| | | Rx | DAY | DAY | DAY |
|---|---|---|---|---|---|
| TIMED TAPPING | BASELINE | DAY 14 | 14-BL | 56 | 56-BL |
| Right Hand “OFF” | 96 | 91 | −5 | 112 | +16 |
| Right Hand “ON” | 110 | 119 | +9 | 100 | −10 |
| Left Hand “OFF” | 76 | 94 | +18 | 69 | −7 |
| Left Hand “ON” | 82 | 79 | −3 | 75 | −7 |

Artane dosage in Case # 6 was variable, perhaps associated with her variable compliance. Chloroquine diphosphate dosage was variable and increased even up to 200 mg, then down to 175 mg. There were significant changes in motor UPDRS scores in the “on” and “off” states compared to baseline, but minimal change in timed tapping scores. As a result, this patient was likely slightly improved with the study medication.

This patient appeared to have mild to severe nausea throughout the study. After 19 days of withdrawal from the study medications, the patient reported that she had declined in mobility, was walking much slower, and was unable to do multiple tasks at one. The patient stated, “I didn't think the medicine was helping me [i.e., during the study medication period], but now I think it was.” The patient expressed a strong desire to resume study medications.

The patient expressed that she felt better and functioned better while taking the study medications. She reported the nausea had not resolved following withdrawal from the study medications. She had thought that the study medication had contributed to her nausea, but now stated that she thought she might need to change her concomitant medications. Approximately 20 days after the study, the patient reported an increase in Parkinson's symptoms, increased tremors, and slower walking.

In the case of Multiple Symptom Atrophy (Case #1), Parkinson's Plus Syndromes (Case #2) and/or other non L-Dopa responsive Atypical Parkinsonian Disorders (Case #3) the compositions comprised of Chloroquine diphosphate with BTAs can be used to effectively improve cognition, alleviate motor symptoms and attenuate the progression of these disorders when administered in dosages similar to dosages that are required to treat Idiopathic Parkinson's Disease. One patient had a confirmed diagnosis of Multiple Symptom Atrophy (MSA). Another patient had received two separate fetal cell transplant procedures (1988 and 1995); yet, enrolled with disabling dyskinesias and exited the study “almost completely without dyskinesia.” Another was newly diagnosed and L-dopa naïve; while, two other patients had PD Plus diagnosis. One patient was medicating incorrectly and therefore experienced no clinical improvement until after treatment day 35, then reported having “come alive.” Another was doing very well on treatment Day 14; but he became intolerant to the study drug following a contraindicated dose increase initiated by a substitute physician in the absence of the Primary Investigator. Three patients had a confirmed diagnosis of idiopathic PD (IPD). All three experienced significant relief in PD symptoms and attenuation in MFs.

Following the treatment period 2-3 week study medication washout period, patients who had reductions made to their concomitant PD medications began experiencing symptom reemergence necessitating dosage increases back to their original pretreatment baseline levels.

This corresponds exceedingly well with CQ's pharmacokinetic properties. Collectively and separately, both CQ enantiomers have extraordinarily long half-lives (t_{1/2}) and mean residence times (MRT): (R) 519−t_{1/2} lambda z=294 hours, MRT=388 hours; (S) 519−t_{1/2} lambda z=236 hours, MRT=372 hours, for instance, as compared to L-dopa: t_{1/2} lambda z=50 minutes or to Sinemet: t_{1/2} lambda z=90 minutes. Following washout, five participants requested to resume taking the study medication. As of our last update, these patients are still taking racemic CQ+BTA, they have remained “stable” (i.e., anti-dyskinetic) and they have continued to do well for over sixteen months.

Our study results demonstrate synergistic interactions between CQ and various other Parkinson's medications. Patients enrolled in the study protocol reported experiencing an attenuation in motor fluctuations, a diminishment of “freezing” and a significant reduction in “OFF” time, while they were taking CQ+BTA. The remarkable consideration was that these improvements were reported to have emerged and were sustained following some very dramatic reductions made in concomitant Parkinson's disease medications.

While the invention has been described in specific terms, it is not to be limited to the description herein but is to be afforded the full scope of the appended claims and all equivalents thereto. For example, other neuromelanin-binding compounds and complexes containing the quinoline ring structure known to the art are equivalent to those specifically described, as are other modifications to the compositions to enhance bioavailability, crossing the blood/brain barrier, biological half-life, or other desirable properties.

1. A method for treating a condition selected from the group consisting of Drug-Induced Dyskinesias, Tardive Dyskinesias, motor fluctuations, cognitive symptoms of Parkinson's disease, Neuroleptic Malignant Syndrome, and negative symptoms of schizophrenia, said method comprising administering to a patient in need of said treatment, in an effective regimen and amount, a composition comprising an active ingredient which is a compound selected from the group consisting of: 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butyl amino) quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof.

2. The method of claim 1 wherein said active ingredient is covalently linked or complexed or mixed with an adjuvant.

3. The method of claim 2 wherein said adjuvant is a peripheral metabolism inhibitor that inhibits peripheral metabolism of said active ingredient.

4. The method of claim 3 wherein said peripheral metabolism inhibitor is an inhibitor of cytochrome P450 2D6 selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, and terbinafine; or a cytochrome P450 3A enzyme inhibitor selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, and norfluoxetine; or racemic mixtures, enantiomers, suitable pharmaceutical salts of the foregoing, or mixtures of any of the foregoing.

5. The method of claim 2 wherein said active ingredient and adjuvant are present in a time-release formula wherein said adjuvant is released about 1.5 to two hours prior to release of said active ingredient.

6. The method of claim 1 wherein said condition is a drug-induced dyskinesia.

7. The method of claim 6 wherein said drug-induced dyskinesia is selected from the group consisting of drug-induced Parkinson's disease, extrapyramidal disorders, akathisia, levodopa-induced dyskinesia, tardive dyskinesia, chorea and ballisms.

8. The method of claim 1 wherein said condition is motor fluctations.

9. The method of claim 1 wherein said movement disorder is selected from the group consisting of idiopathic Parkinson's disease, multiple symptom atrophy associated with Parkinson's disease, Parkinson's Plus Syndrome, Atypical Parkinsonian Disorders, on-off syndrome associated with treatment with dopamine or a dopamine agonist, conditions characterized by nigrostriatal degeneration, vascular Parkinson's disease, Huntington's Chorea, and Wilson's Disease.

10. The method of claim 1 wherein said condition is a negative symptom of schizophrenia.

11. The method of claim 10 wherein said negative symptom of schizophrenia is selected from the group consisting of apathy, loss of verbal fluency, affective flattening, lack of motivation, and depression.

12. The method of claim 1 wherein said condition is Neuroleptic Malignant Syndrome.

13. The method of claim 1 wherein said condition is a cognitive symptom of Parkinson's disease selected from the group consisting of memory loss and loss of ability to multi-task.

14. A method for inhibiting oxidative stress responsible for negative symptoms of schizophrenia in a patient suffering from negative symptoms of schizophrenia, said method comprising administering to said patient, in an effective regimen and amount, a composition comprising an active ingredient which is a compound selected from the group consisting of: 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylarnino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof.

15. The method of claim 14 wherein said active ingredient is covalently linked or complexed or mixed with an adjuvant.

16. The method of claim 15 wherein said adjuvant is a peripheral metabolism inhibitor that inhibits peripheral metabolism of said active ingredient.

17. The method of claim 16 wherein said peripheral metabolism inhibitor is an inhibitor of cytochrome P450 2D6 selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, and terbinafine; or a cytochrome P450 3A enzyme inhibitor selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, and norfluoxetine; or racemic mixtures, enantiomers, suitable pharmaceutical salts of the foregoing, or mixtures of any of the foregoing.

18. The method of claim 15 wherein said active ingredient and adjuvant are present in a time-release formula wherein said adjuvant is released about 1.5 to two hours prior to release of said active ingredient.

19. A method for reducing apoptosis in melanized catecholamine neurons in a patient suffering from a degenerative condition of said neurons, said method comprising administering to said patient, in an effective regimen and amount, a composition a composition comprising an active ingredient which is a compound selected from the group consisting of: 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof.

20. The method of claim 19 wherein said active ingredient is covalently linked or complexed or mixed with an adjuvant.

21. The method of claim 20 wherein said adjuvant is a peripheral metabolism inhibitor that inhibits peripheral metabolism of said active ingredient.

22. The method of claim 21 wherein said peripheral metabolism inhibitor is an inhibitor of cytochrome P450 2D6 selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, and terbinafine; or a cytochrome P450 3A enzyme inhibitor selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, and norfluoxetine; or racemic mixtures, enantiomers, suitable pharmaceutical salts of the foregoing, or mixtures of any of the foregoing.

23. The method of claim 20 wherein said active ingredient and adjuvant are present in a time-release formula wherein said adjuvant is released about 1.5 to two hours prior to release of said active ingredient.

24. A method for selectively increasing glial-derived neurotrophic factor (GDNF) in the substantia nigra, striatum and/or globus pallidus of a patient suffering from a condition characterized by striatal neural degeneration, said method comprising administering to said patient, in an effective regimen and amount, a composition a composition comprising an active ingredient which is a compound selected from the group consisting of: 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy-′-bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof.

25. The method of claim 24 wherein said active ingredient is covalently linked or complexed or mixed with an adjuvant.

26. The method of claim 25 wherein said adjuvant is a peripheral metabolism inhibitor that inhibits peripheral metabolism of said active ingredient.

27. The method of claim 26 wherein said peripheral metabolism inhibitor is an inhibitor of cytochrome P450 2D6 selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, and terbinafine; or a cytochrome P450 3A enzyme inhibitor selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, and norfluoxetine; or racemic mixtures, enantiomers, suitable pharmaceutical salts of the foregoing, or mixtures of any of the foregoing.

28. The method of claim 25 wherein said active ingredient and adjuvant are present in a time-release formula wherein said adjuvant is released about 1.5 to two hours prior to release of said active ingredient.

29. A method for reducing thalamic hyperactivity in a patient having a condition associated with thalamic hyperactivity, said method comprising administering to said patient, in an effective regimen and amount, a composition a composition comprising an active ingredient which is a compound selected from the group consisting of: 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof.

30. The method of claim 29 wherein said active ingredient is covalently linked or complexed or mixed with an adjuvant.

31. The method of claim 30 wherein said adjuvant is a peripheral metabolism inhibitor that inhibits peripheral metabolism of said active ingredient.

32. The method of claim 31 wherein said peripheral metabolism inhibitor is an inhibitor of cytochrome P450 2D6 selected from the group consisting of amiodarone, celecoxib, chlorpheniramine, cimetidine, clomipramine, fluoxetine, levomepromazine, metoclopramide, mibefradil, moclobemide, paroxetine, quinidine, ranitidine, ritonavir, sertraline, and terbinafine; or a cytochrome P450 3A enzyme inhibitor selected from the group consisting of delaviridine, indinavir, nelfinavir, saquinavir, amiodarone, cimetidine, ciprofloxacin, clarithromycin, diethyl-dithiocarbamate, diltiazem, erythromycin, fluconazole, fluvoxamine, itraconazole, ketoconazole, mifepristone, nefazodone, norfloxacinem, and norfluoxetine; or racemic mixtures, enantiomers, suitable pharmaceutical salts of the foregoing, or mixtures of any of the foregoing.

33. The method of claim 30 wherein said active ingredient and adjuvant are present in a time-release formula wherein said adjuvant is released about 1.5 to two hours prior to release of said active ingredient.

34. A therapeutic composition comprising a first active ingredient selected from the group consisting of 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-methylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy--bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof; and
a second active ingredient which is dopamine or a dopamine precursor or dopamine agonist; and
wherein said first active ingredient and said second active ingredient are present at a ratio between about 5:95 to about 25:75.

35. A therapeutic composition comprising a first active ingredient selected from the group consisting of 7-chloro-4-(4-diethylamino-1-methylbutylamino)quinoline (chloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-chloro-4-(4-ethyl-(2-hydroxyethyl)-amino-1-methylbutylamino)quinoline (hydroxychloroquine); 7-fluoro-4-(4-ethyl-(2-hydroxyethyl)-amino-1-methylbutylamino)quinoline; 4-(4-ethyl-(2-hydroxyethyl)-amino-1-m ethylbutylamino)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino)-6-methoxydihydrochloride quinoline; 1-acetyl-1,2,3,4-tetrahydroquinoline; 8-[4-aminopentyl)amino]-6-methoxyquinoline dihydrochloride; 1-butyryl-1,2,3,4-tetrahydroquinoline; 3-chloro-4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3-fluoro-4-(4-hydroxy--bis(2-methyl-1-pyrrolidinyl)-2,5-xylidinoquinoline, 4-[(4-diethylamino)-1-methylbutyl)amino]-6-methoxyquinoline; 4-(4-hydroxy-,′-bis(2-methyl-1-pyrrolidinyl)-2,5-xylidinoquinoline, 4-[(4-diethylamino)-1-methylbutyl)amino]-6-methoxyquinoline; 3,4-dihydro-1-(2H)-quinolinecarboxyaldehyde; 1,1′-pentamethylenediquinoleinium diiodide; and 8-quinolinol sulfate, racemic mixtures, and enantiomers thereof, phosphate salts and other suitable pharmaceutical salts thereof, and mixtures thereof; and
a second active ingredient which is a dopamine antagonist.

36. The composition of claim 35 wherein said dopamine antagonist is selected from the group consisting of Chlorpromazine, Chlorprothixene, Fluphenazine, Haloperidol, Loxapine, Mesoridazine, Molindone, Perphenazine, Pimozide, Prochlorperazine, Promazine, Thioridazine, Thiothixene, Trifluoperazine, Fluphenazine decanoate and Haloperidol decanoate.
